SEQUENCE LISTING (2 sequences)

SEQ ID NO: 1 - DNA, 84 bp, synthetic construct
  source 1..84: mol_type = other DNA
gggaatttccggggactttccgggaatttccggggactttccgggaatttcctagagggtatataatggaagctcgacttccag

SEQ ID NO: 2 - DNA, 21 bp, synthetic construct
  source 1..21: mol_type = other DNA
caaagatgggatgagaaagga

(19) United States
(12) Patent Application Publication
(10) Pub. No.: US 2026/0265774 A1
(43) Pub. Date: Sep. 10, 2026

(54) RECOMBINANT ADENO-ASSOCIATED VIRUS FOR THE TREATMENT OF INFLAMMATORY DISEASES, CONSTRUCTION METHODS AND USES THEREOF

(71) Applicant: SOUTHEAST UNIVERSITY, Nanjing, Jiangsu (CN)

(72) Inventors: Jinke WANG, Nanjing (CN); Tao LUO, Nanjing (CN); Hailin TANG, Nanjing (CN)

(73) Assignee: SOUTHEAST UNIVERSITY, Nanjing, Jiangsu (CN)

(21) Appl. No.: 18/847,223

(22) PCT Filed: Mar. 14, 2023
(86) PCT No.: PCT/CN2023/081330

§ 371 Date: Dec. 20, 2024

(30) Foreign Application Priority Data
Mar. 14, 2022 (CN) 202210249034.9

(51) Int. Cl. C12N 15/86; A61P 29/00; C12N 15/113

(52) U.S. Cl. CPC C12N 15/86; A61P 29/00; C12N 15/113; C12N 2310/141; C12N 2750/14143; C12N 2750/14151

(57) ABSTRACT

A recombinant adeno-associated virus for the treatment of inflammatory diseases, and a construction method and a use thereof are provided. The recombinant adeno-associated virus uses an adeno-associated virus as a vector and contains one or more copies of a functional DNA fragment DMP-miR533. The functional DNA fragment DMP-miR533 is composed of two functional elements DMP and miR533. The DMP is an NF-κB specific promoter, and the miR533 is a microRNA that targets an NF-κB mRNA. The rAAV-DMP-miR533 of the present disclosure has a good therapeutic effect on acute colitis induced by sodium dextran sulfate, psoriasis induced by imiquimod, and arthritis induced by collagen in mice, and has good safety in the treatment of these inflammatory model mice. The rAAV-DMP-miR533 is expected to provide a new technology and new agent for the treatment of various inflammatory diseases.

### TECHNICAL FIELD

The present invention relates to the biotechnical field of inflammatory gene therapy, and in particular to a recombinant adeno-associated virus and its application in the treatment of inflammatory diseases.

### BACKGROUND

Inflammation is the body's protective response to infection and physical damage. A moderate inflammatory response helps maintain homeostasis in the body. However, abnormal inflammatory responses can lead to inflammatory diseases. For example, some infectious pathogens can cause systemic inflammation, which may lead to sepsis, cytokine release syndrome, acute respiratory distress syndrome, and even multiple organ failure. Some persistent infections can lead to chronic inflammation and a high risk of cancer, such as hepatitis and liver cancer caused by hepatitis B virus. Many chronic inflammations are caused by abnormal changes in adaptive immunity, leading to various autoimmune diseases such as arthritis, inflammatory bowel disease, lupus, psoriasis, dermatitis, asthma, multiple sclerosis, steatohepatitis, and even atherosclerosis, diabetes, neurodegenerative diseases, and inflamm-aging. In addition, many abnormal changes in innate immunity can also induce various autoinflammatory diseases. Therefore, abnormal inflammation poses a wide and serious threat to human health.

In order to treat inflammatory diseases, the molecular mechanisms associated with these diseases have been widely studied. At present, their basic signaling pathways (e.g., NF—KB and JAK-STAT) and the main involved molecules have been identified, and many potential targets have been tried for the development of anti-inflammatory drugs. To date, many drugs have been used to treat various inflammatory diseases, such as widely used corticosteroids (e.g., glucocorticoids). Meanwhile, many anti-inflammatory biological agents have also been developed, such as cytokine monoclonal antibodies (e.g., proinflammatory cytokines TNF-α, IL-1α, IL-1β, IL-5, IL-6, IL-12, IL-17A, IL-17F, IL-23, and anti-inflammatory cytokines IL-4, IL-10, IL-11, IL-13, TGFβ), antibodies or antagonists of cytokine receptors (e.g., IL-6R, IL-5Rα, IL-4Rα), and CD antibodies (e.g., CD4, CD14, CD19, CD20, CD38, CD40). JAK small molecule inhibitors (JAK1, JAK2, JAK3, TYK2) are rapidly developing promising new anti-inflammatory drugs. There is no doubt that current anti-inflammatory drugs have benefited patients greatly. However, anti-inflammatory drugs still face several key challenges, such as primary non-response, drug resistance, or loss of response, recurrence, and multiple side effects. Therefore, current treatment methods are far from meeting the needs of clinical treatment of inflammatory diseases.

NF-κB is a family of sequence-specific DNA-binding transcription factors, including RelA/p65, p50, p52, RelB and c-Rel, which play a key regulatory role in inflammation. After being induced by various inducers, activated NF-κB (mainly RelA-p50 heterodimers) can induce the expression of target genes by directly binding to promoters and enhancers, thereby participating in processes such as cell proliferation, apoptosis, and innate immune response. NF-κB can directly regulate the expression of multiple inflammatory genes, including adhesion factors (e.g., ICAM-1, and VCAM-1), cytokines (e.g., IL-1α, IL-1β, IL-2, IL-6, IL-8, IL-10, IL-11, IL-12, IL-13, IL-15, IL-17, IL-23, TNFα, IFNβ, and IFNγ), and chemokines (e.g., CCL5, CCL17, CCL19, CCL20, CCL22, CCL23, and CCL28). Decoy oligonucleotides and small interfering nucleic acids (siRNA) have also been used as candidate inhibitors of NF-κB. However, due to the uncontrollable activity, difficult delivery, and easy degradation, these two NF-κB inhibitors have also failed in clinical trials. In order to overcome the limitations, the applicant previously developed a new NF-κB inhibitor molecule: a plasmid vector DMP-miR533 (Int. J. Biochem. Cell. Biol. 2018, 95:43-52; Patent No. ZL201710812983.2) that combines NF-κB decoy and microRNA sequence.

In the prior art of the applicant, it has been preliminarily demonstrated on cells cultured in vitro that the DMP-miR533 plasmid vector can sense and inhibit the activity of NF-κB in cells, but the plasmid vector itself cannot be directly used for animal administration at present, which means that this kind of plasmid vector cannot achieve the transfection of cells in vivo and the control of NF-κB activity. In addition, inflammation is not the behavior of a certain cell in vitro, but a physiological or pathological reaction at the level of living mammals. Therefore, it is still unknown whether DMP-miR533, which can sense and control the activity of NF-κB in cells cultured in vitro, can control the activity of NF-κB in living mammals and achieve the treatment of inflammation. In addition, cells cultured in vitro are a very simple artificial controllable environment, which cannot be compared with the physiological and pathological environment in vivo of living mammals with complex life systems. This is why many promising candidate drug molecules fail to become drugs in clinical trials. Therefore, in order to demonstrate and develop the possibility of DMP-miR533 controlling NF-κB activity in living mammals and achieving the treatment of inflammation, new technologies are explored for the treatment of human inflammation.

### SUMMARY

Objective of the present disclosure: in view of the problems existing in the current treatment of inflammation, the present disclosure provides a novel recombinant adeno-associated virus, which can achieve the treatment of inflammatory diseases in living mammals by inhibiting the activity of intracellular NF-κB, and is expected to be used for the preparation of novel biological drugs for the treatment of human inflammatory diseases.

The present disclosure further provides a construction method and a use of the recombinant adeno-associated virus.

Technical solution: in order to achieve the objective, the present disclosure provides a recombinant adeno-associated virus for the treatment of inflammatory diseases. A recombinant adeno-associated virus rAAV-DMP-miR533 uses an adeno-associated virus as a vector and contains one to multiple copies of a functional DNA fragment DMP-miR. The functional DNA fragment DMP-miR is composed of two functional elements DMP and miR, wherein the DMP is an NF-κB specific promoter, and the miR is a microRNA that targets an NF-κB mRNA.

The DMP is a NF-κB specific promoter composed of an NF-κB decoy and a minimal promoter. The DMP includes NF-κB decoys and minimal promoters of various sequences.

Preferably, the DMP has a sequence of 5′-GGG AAT TTC CGG GGA CTT TCC GGG AAT TTC CGG GGA CTT TCC GGG AAT TTC CTA GAG GGT ATA TAA TGG AAG CTC GAC TTC CAG-3′ (SEQ ID NO. 1).

The miR encodes an artificial microRNA targeting an NF-κB family member RELA, RELB or CREL.

Preferably, the miR encodes an artificial microRNA targeting the NF-κB family member RELA. Preferably, the miR encodes an miR533, wherein the miR533 has a sequence of 5′-CAA AGA TGG GAT GAG AAA GGA-3′ (SEQ ID NO.2).

After the functional DNA fragment DMP-miR is introduced into a cell by the recombinant adeno-associated virus, the functional element DMP is capable of binding to a transcription factor protein NF-κB in a nucleus of the cell to activate the expression of miR.

Furthermore, the expressed miR533 is capable of binding to the NF-κB mRNA in a cytoplasm after being processed and matured by an intracellular microRNA maturation system, thereby inhibiting the expression of an NF-κB protein. The processing and maturation by the intracellular microRNA maturation system means that the initially expressed miR must be cleaved and processed by these systems (some proteins and complexes thereof) to become mature miR before it can exert the function. The processing include an initial transcription product of miR, pri-miRNA, being processed into pre-miRNA by a Drosh-DGCR8 complex in the cell nucleus; the pre-miRNA entering the cytoplasm from the cell nucleus with the assistance of an Exportin 5 protein; and the pre-miRNA being further processed into miRNA by a Dicer-TRBP complex in the cytoplasm.

The adeno-associated virus includes any one of various serotypes of adeno-associated viruses, such as any one of AAV1-AAV9.

Preferably, the adeno-associated virus is AAV2.

The construction method of the recombinant adeno-associated virus for the treatment of inflammatory diseases of the present disclosure comprises the following steps:
(1) amplifying the DMP-miR533 from a vector pDMP-miR533 and ligating the DMP-miR533 to a vector pAAV-MCS to construct pAAV-DMP-miR533;
(2) transfecting a 293T cell with the pAAV-DMP-miR533 and two helper plasmids (pAAV-Helper and pAAV-RC; Stratagene); after cell culture, collecting the cell and a culture medium culturing the cell and freeze-thawing the cell and the culture medium, adding pure chloroform to a lysate of the freeze-thawed cell to obtain a mixture and shaking the mixture; adding NaCl to the mixture, shaking the mixture until the NaCl is dissolved, and collecting a supernatant after centrifugation; adding PEG8000 to the supernatant, shaking it until the PEG8000 is dissolved, and discarding a supernatant after centrifugation, dissolving a precipitate, adding DNase and RNase to the dissolved precipitate to react and incubating at a room temperature to obtain a reactant, extracting the reactant to collect an aqueous phase containing a purified virus, and after the virus is quantified, segmenting the aqueous phase containing the purified virus and storing at −80° C. for later use, wherein the obtained virus is named as the rAAV-DMP-miR533.

The present disclosure provides a use of a recombinant adeno-associated virus for the treatment of inflammatory diseases in the preparation of a novel agent for the treatment of inflammatory diseases.

In some embodiments, the inflammatory diseases include various types of inflammation-related diseases, such as inflammation caused by infection, spontaneous inflammation, autoimmune diseases, neurodegenerative diseases, cancer, etc.

The inflammatory diseases include acute colitis, psoriasis, and arthritis.

The rAAV-DMP-miR533 of the present disclosure can be used to prepare a novel safe anti-inflammatory agent that can be administered in various ways.

Preferably, the rAAV-DMP-miR533 can be mixed with agents such as petrolatum (Vaseline®) to prepare an external application agent or medicine for the treatment of skin inflammation such as psoriasis. This administration method greatly facilitates the treatment of local skin inflammation.

The present disclosure provides a recombinant adeno-associated virus rAAV-DMP-miR533. The DMP is an NF-κB specific promoter formed by connecting an NF-κB decoy and a minimal promoter. The miR533 can encode an artificial microRNA targeting NF-κB RELA. Experimental studies have shown that the rAAV-DMP-miR533 virus has a good therapeutic effect on acute colitis induced by sodium dextran sulfate, psoriasis induced by imiquimod, and arthritis induced by collagen in mice, and has good safety in the treatment of these inflammatory mouse models. The rAAV-DMP-miR533 is expected to provide a new technology and new agent for the treatment of various inflammatory diseases.

The DMP in the present disclosure is an NF-κB specific promoter composed of an NF-κB decoy and a minimal promoter. The MiR533 is an artificial microRNA targeting NF-κB RELA. It has been previously demonstrated that the DMP-miR533 can sense and control NF-κB activity in cultured cells in vitro. Transfection of the DMP-miR533 can cause apoptosis of cells with over-activated NF-κB, but has little effect on normal cells. In the present disclosure, the DMP-miR533 is packaged in an adeno-associated virus (AAV) to solve the in vivo delivery problem, and a recombinant AAV (rAAV) packaged with DMP-miR533 is used to demonstrate whether the DMP-miR533 has the effect of controlling inflammation in vivo and whether it is safe. In the present disclosure, the inhibitory effect of rAAV-DMP-miR533 on in vitro inflammatory cells was first evaluated in vitro, demonstrating the successful packaging of rAAV-DMP-miR533, the ability to infect cells, and the inhibition of over-activated NF-κB activity in cells. Then, rAAV-DMP-miR533 was used to treat mice with three typical inflammatory diseases, including acute colitis induced by dextran sulfate sodium (DSS), psoriasis induced by imiquimod (IMQ), and arthritis induced by collagen. This demonstrates the feasibility, reliability, and safety of rAAV-DMP-miR533 in the treatment of inflammation in living mammals.

In terms of mechanism, the present disclosure found that rAAV-DMP-miR533 can cause apoptosis of inflammatory cells, thereby eradicating inflammatory cells. Inflammatory cells can secrete proinflammatory cytokines to exacerbate and worsen the inflammatory process. rAAV-DMP-miR533 can remove the source of inflammation by eradicating inflammatory cells. This mechanism is different from the current anti-inflammatory strategy that relies on antibodies or antagonists of cytokines and receptors thereof. Most of the direct target genes of NF-κB are cytokines, while the inhibition of cytokines with antibodies can only transiently neutralize the produced cytokines, and cannot eradicate the inflammatory source that produces cytokines. This results in inflammatory cells still being able to continue to produce cytokines. Therefore, the recurrence rate of inflammatory diseases treated with cytokine antibodies is high. In addition, the pleiotropic and redundant combination of many cytokines with their receptors limits the efficacy of a single neutralizing agent, which is the reason why inflammatory diseases are resistant to current treatment strategies. It is critical that cytokines that are key players or key regulators of specific inflammatory diseases may vary from person to person, resulting in low patient response rates for most current treatments. In contrast, rAAV-DMP-miR533 inhibited all cytokines associated with inflammation by eradicating inflammatory cells. For example, in three inflammatory mouse models, rAAV-DMP-miR533 significantly reduced the levels of two major proinflammatory factors, TNF-α and IL-6, in serum at both the mRNA and protein levels. This reduction in the levels of major inflammatory factors in the blood is extremely important for treating local inflammatory symptoms and eliminating the systemic pathological effects of inflammatory factors. Therefore, rAAV-DMP-miR533 provides a new strategy for broader anti-inflammatory effects, which may overcome some of the key challenges of current anti-inflammatory treatments, such as low response, drug resistance, recurrence, and side effects.

Beneficial effects: compared with the prior art, the present disclosure has the following advantages:

The current agents for the treatment of inflammation are mainly antibodies to various cytokines (e.g., TNF-α antibodies) and emerging JAK small molecule inhibitors. These therapeutic agents have the key clinical problems of low response rate and drug resistance. In order to break through the existing technology for the treatment of inflammation, a new inflammatory gene therapy technology based on AAV is developed. In the present disclosure, the DNA fragment DMP-miR533 invented by the applicant in the early stage is packaged into a safe gene delivery vector AAV to produce a new AAV particle-rAAV-DMP-miR53. The anti-inflammatory effect of rAAV-DMP-miR533 at the cellular and in vivo levels was systematically studied. The results show that rAAV-DMP-miR533 has excellent anti-inflammatory effects both in vitro and in vivo. In particular, the rAAV demonstrates good treatment effects to inflammation in three typical inflammatory mouse models, including an acute colitis model induced by dextran sulfate sodium (DSS) in mice, a psoriasis model induced by imiquimod (IMQ), and a mouse arthritis model induced by collagen. In addition, during the in vivo treatment process, the rAAV-DMP-miR533 also showed good biosafety. Therefore, the rAAV-DMP-miR53 developed by the present disclosure is expected to be used to prepare new biological drugs for the treatment of human inflammatory diseases.

Compared with traditional NF-κB small molecule inhibitors, decoys, and siRNA, the rAAV-DMP-miR533 has the advantages that the rAAV-DMP-miR533 can avoid excessive inhibition of NF-κB activity in normal cells, which is the fundamental reason why traditional NF-κB small molecule inhibitors, decoys, and siRNA cannot become drugs. For example, BAY 11-7082, a typical small molecule NF-κB inhibitor, causes significant apoptosis in both inflammatory cells and normal cells. The experimental study of the present disclosure shows that the rAAV-DMP-miR533 can induce significant apoptosis in cancer cells with over-activated NF-κB (a typical inflammatory cell), but has almost no effect on normal cells. However, when normal cells are induced by NF-κB inducer TNF-α to become inflammatory cells, the rAAV-DMP-miR533 can induce apoptosis of inflammatory normal cells, which shows that the rAAV-DMP-miR533 can specifically act on inflammatory cells, and the high selectivity is of great significance for improving in vivo application and reducing side effects. Therefore, the rAAV-DMP-miR533 overcomes the side effects of traditional NF-κB inhibitors and has potential for clinical translation.

AAV is a safe gene delivery tool with the advantages of low immunogenicity, no pathogenicity, no genome insertion, long-term stable expression, etc., and has been approved for human clinical gene therapy. The experimental study of the present disclosure shows that under the premise of the used dose, the rAAV-DMP-miR533 has no biological toxicity to mice with three inflammatory diseases. In particular, three high-dose intravenous administrations were performed on arthritis CIA mice, and the rAAV-DMP-miR533 had no effect on the serum biochemical indicators and spleen of mice. In contrast, the widely used anti-arthritis drug MTX showed significant toxic side effects on the liver and spleen of mice. In addition, in the present disclosure, it was discovered for the first time that the rAAV-DMP-miR533 can be mixed with petrolatum for external application to treat psoriasis. This mode of administration greatly facilitates the treatment of local skin inflammation, which has not been reported internationally and is an important innovation in the route of administration of rAAV gene therapy. Therefore, the rAAV-DMP-miR533 is a new type of safe anti-inflammatory agent that can be administered in various ways. The potential clinical application of the rAAV-DMP-miR533 is still challenged by pre-existing or in vivo antibodies that can neutralize AAV vectors as these antibodies can block the administration and re-administration of AAV, which is also a common obstacle to current AAV-based therapies. However, the obstacle can be overcome by some new methods, such as using endopeptidases (e.g., Imlifidase, and IdeS) or Immunoglobulin-degrading enzyme from Streptococcus equi subspecies zooepidemicus (IdeZ) to eliminate pre-existing anti-AAV antibodies, using CRISPR to transiently inhibit endogenous Myd88, and using AAV modified vectors with TLR9 inhibitory sequences. In addition, other non-viral vectors (e.g., lipid nanoparticles (LNPs)) can also be used for the delivery of DMP-miR533. It is believed that the empty shell rAAV remaining in the rAAV packaging may also be an effective rAAV antibody neutralizer.

In the present disclosure, AAV-based gene therapies also developed for the treatment of inflammatory diseases. At present, the AAV-based gene therapies are mainly used for the treatment of human genetic diseases. Some AAV-based gene therapies have been carried out in clinical trials, such as the treatment of inflammatory diseases by expressing fusion proteins of IFN-β or TNFR-IgG1 Fc, especially autoimmune diseases (e.g., rheumatoid arthritis). However, these treatments still only target one inflammation-related cytokine. In contrast, the rAAV-DMP-miR533 directly targets the inflammatory center NF-κB. The treatment of three typical inflammatory diseases shows that the rAAV-DMP-miR533 based on the AAV gene therapy provides broader treatment strategies for inflammatory diseases.

### BRIEF DESCRIPTION OF THE DRAWINGS

FIG. 1 is a schematic diagram illustrating plasmids and viral vectors, including plasmid maps of pAAV-MCS, pAAV-DMP-NT, pAAV-DMP-miR533, pAAV-CMV-EGFP, and pAAV-DMP-miR533-CMV-EGFP, and packaged recombinant AAVs (rAAVs) thereof. The prepared rAAVs are named rAAV-MCS, rAAV-DMP-NT, rAAV-DMP-miR533, rAAV-CMV-EGFP, and rAAV-DMP-miR533-CMV-EGFP, respectively.

FIG. 2 is a schematic diagram illustrating inflammation treatment with rAAV-DMP-miR533. (A) A schematic diagram illustrating inflammation treatment with rAAV-DMP-miR533. DMP, decoy-minimal promoter; Pol II, RNA polymerase II; RISC, RNA-induced silencing complex. (B) Expression levels of NF-κB in inflammatory cells and normal cells. Cancer cells HT-29 and CT-26 with NF-κB activity are considered natural inflammatory cells. Normal cells HL7702 and NIH-3T3 without NF-κB activity. However, when induced with the proinflammatory cytokine TNF-α, the cancer cells HT-29 and CT-26 with NF-κB activity and the normal cells HL7702 and NIH-3T3 without NF-κB activity became induced inflammatory cells with NF-κB activity. The expression of NF-κB RELA was detected by qPCR (n=3 wells).

FIG. 3 is a schematic diagram illustrating the treatment of inflammatory cells with pAAV-DMP-miR533. HT-29 cells were transfected with various plasmids and then cultured for 24, 48, and 72 h, respectively. (A) Representative fluorescence images of HT-29 cells after acridine orange (OA) staining. Scale bar: 100 μm. (B) Live cells counted at different time points (n=3 images). Cell counting was performed using Image-Pro Plus software on fluorescence images after acridine orange staining. (C) Growth curve of HT-29 cells detected by CCK-8 (n=3 wells). (D) Relative expression of NF-κB and target genes thereof in HT-29 cells was detected by qPCR 48 h after transfection with various plasmids (n=3 wells). RQ=2-AACt. RQ, relative quantification. (E) Representative fluorescence images of HL7702 cells after acridine orange staining. Scale bar: 100 μm. HL7702 cells were induced with TNF-α and then transfected with plasmids as needed. Transfected cells were cultured for 24, 48, and 72 h, respectively. Blank group, MCS, and miR533: cells transfected with Lipofectamine, pAAV-MCS, and pAAV-DMP-miR533, respectively; TNF-α: cells induced with TNF-α (cells were induced with TNF-α at a final concentration of 10 ng/ml for 1 h); TNF-α+MCS and TNF-α+miR533: cells induced with TNF-α after transfection with pAAV-MCS and pAAV-DMP-miR533, respectively.

FIG. 4 is a schematic diagram illustrating the treatment of inflammatory cells with rAAV-DMP-miR533-CMV-EGFP. HL7702 cells were induced with TNF-α as needed and then infected with various rAAVs for 48 h. (A) Cell fluorescence images. Scale bar: 100 μm. (B) Cell fluorescence intensity analyzed by flow cytometry (n=3 wells). (C) Flow cytometry analysis of representative cell apoptosis. (D) Cell apoptosis analyzed by flow cytometry (n=3 wells). (E) Cell viability detected by CCK-8 (n=3 wells). (F) Expression of NF-κB and target genes thereof in cells 48 h after virus infection was detected by qPCR (n=3 wells). Blank group, MCS, miR533, EGFP and miR533-EGFP: cells were infected with phosphate buffered saline (PBS), rAAV-MCS, rAAV-DMP-miR533, rAAV-CMV-EGFP, and rAAV-DMP-miR533-CMV-EGFP, respectively; TNF-α: cells induced by TNF-α (cells were induced for 1 h with TNF-α at a final concentration of 10 ng/ml); TNF-α+MCS, TNF-α+miR533, TNF-α+EGFP, TNF-α+miR533-EGFP: cells induced by TNF-α were infected with rAAV-MCS, rAAV-DMP-miR533, rAAV-CMV-EGFP, and rAAV-DMP-miR533-CMV-EGFP, respectively.

FIG. 5 is schematic diagram illustrating a flow cytometric analysis of EGFP fluorescence intensity. HL7702 cells were induced with or without TNF-α at a final concentration of 10 ng/mL for 1 h and then infected with various viruses for 48 h. MCS, miR533, EGFP and miR533-EGFP: cells were infected with phosphate buffered saline (PBS), rAAV-MCS, rAAV-DMP-miR533, rAAV-CMV-EGFP, and rAAV-DMP-miR533-CMV-EGFP, respectively; TNF-α+MCS, TNF-α+miR533, TNF-α+EGFP, TNF-α+miR533-EGFP: cells induced by TNF-α were infected with rAAV-MCS, rAAV-DMP-miR533, rAAV-CMV-EGFP and rAAV-DMP-miR533-CMV-EGFP, respectively.

FIG. 6 is schematic diagram illustrating the treatment of colitis mice with rAAV-DMP-miR533. The colitis mouse model was established by dextran sulfate sodium (DSS) induction and treated by intravenous injection of rAAVs (iv). (A) A schematic diagram illustrating the establishment and treatment of the DSS-induced acute colitis mouse model. (B) Blood stains around the anus of the mouse. (C) Body weight of the mouse. (D) Colon of the mouse. (E) Length of the colon of the mouse (n=6 mice). (F) H&E stained sections of representative colon samples. Black boxes indicate magnified areas. Scale bars: 200 μm (10×) and 100 μm (20×). (G) Histopathological scores of colon samples (n=6 mice). (H) ELISA detection of TNF-α and IL-6 levels in serum (n=6 mice). (I) qPCR detection of the expression of NF-κB RELA and target genes thereof in colon samples (n=6 mice). Blank group, mice drank normal water and were treated with PBS; DSS group, mice drank water containing 3% DSS (DSS-induced mice) and were treated with PBS; MCS group, DSS-induced mice were treated with rAAV-MCS; miR533, DSS-induced mice were treated with rAAV-DMP-miR533. ns, no significant difference.

FIG. 7 is a schematic diagram illustrating the establishment of a colitis mouse model by dextran sulfate sodium (DSS) induction and treatment by intravenous injection of rAAVs (iv) (biological replicate 2). (A) A schematic diagram illustrating the establishment and treatment of the DSS-induced acute colitis mouse model. (B) Blood stains around the anus of the mouse. (C) Body weight of the mouse. (D) Colon of the mouse. (E) Length of the colon of the mouse (n=6 mice). (F) H&E-stained sections of representative colon samples. Black boxes indicate magnified areas. Scale bars: 200 μm (10×) and 100 μm (20×). (G) Histopathological scores of colon samples (n=6 mice). Blank group, mice drank normal water and were treated with PBS; DSS group, mice drank water containing 3% DSS (DSS-induced mice) and were treated with PBS; MCS group, DSS-induced mice were treated with rAAV-MCS; miR533, DSS-induced mice were treated with rAAV-DMP-miR533. (H) ELISA detection of the levels of TNF-α and IL-6 in serum (n=6 mice).

FIG. 8 is a schematic diagram illustrating the treatment of psoriasis mice by intravenous injection of rAAV-DMP-miR533. The psoriasis mouse model was established by imiquimod (IMQ) induction and treated by intravenous injection of rAAVs (iv). (A) A schematic diagram illustrating the construction and treatment of the psoriasis mouse model. (B) An intuitive diagram of the back skin of the mouse. (C) H&E stained sections of representative skin samples. Scale bars: 200 μm (10×) and 100 μm (20×). (D) Histopathological scores of skin samples 6 days after IMQ induction (n=3 mice). (E) Histopathological scores of skin samples 12 days after IMQ induction (blank group and MCS group, n=3 mice; miR533 group, n=6 mice). (F) ELISA detection of the levels of TNF-α and IL-6 in serum (blank group and MCS group, n=3 mice; miR533 group, n=6 mice). (G) qPCR detection of TNF-α and IL-6 mRNA levels in skin samples (blank and MCS groups, n=3 mice; miR533 group, n=6 mice). Blank group, mice treated with PBS and smeared with vaseline; MCS, mice treated with rAAV-MCS and induced with IMQ; miR533, mice treated with rAAV-DMP-miR533 and induced with IMQ. ns, no significant difference.

FIG. 9 is a schematic diagram illustrating H&E pathological analysis and gene expression detection of relevant tissues in IMQ-induced psoriasis mice after rAAV-DMP-miR533 tail vein injection. (A) H&E stained sections of all skin samples in different groups (blank group, MCS, miR533). (B) Expression of NF-κB and target genes thereof in skin samples detected by qPCR.

FIG. 10 is a schematic diagram illustrating the efficacy of rAAV-DMP-miR533 in a psoriasis mouse model by subcutaneous injection (ih.) and skin administration (ad us. ext.) (topical application). These experiments were performed using only one mouse. (A) Images of the back skin of the mouse on days 0, 6, and 12, respectively. (B) Typical H&E-stained tissue sections of skin samples. (C) ELISA detection of TNF-α and IL-6 in serum. (D) qPCR detection of the expression of NF-κB and target genes thereof in skin samples. F, subcutaneous injection (ih.); T, skin administration (ad us. ext.).

FIG. 11 is a schematic diagram illustrating the treatment of psoriasis mice by topical skin administration of rAAV-DMP-miR533. The psoriasis mouse model was established by imiquimod (IMQ) induction and rAAVs were administered by skin application (topical application). (A) A schematic diagram of the construction and treatment of the psoriasis mouse model. (B) Visual images of the back skin of the mouse. (C) Psoriasis area and severity index (PASI) score (n=6 mice). (D) H&E stained sections of representative skin samples. Scale bars: 200 μm (10×) and 100 μm (20×). (E) Histopathological score of skin samples (n=6 mice). (F) Visual image of mouse spleen. (G) Spleen weight (n=6 mice). (H) Average weight of mice in each group (n=6 mice). (I) ELISA detection of TNF-α and IL-6 levels in serum (n=6 mice). Blank group, mice treated with PBS and coated with vaseline; MCS, mice treated with rAAV-MCS and induced with IMQ; miR533, mice treated with rAAV-DMP-miR533 and induced with IMQ. ns, not significant.

FIG. 12 is a schematic diagram illustrating the treatment of imiquimod (IMQ)-induced psoriasis mouse model by skin application of rAAV-DMP-miR533. (A) H&E stained sections of all skin samples in different groups (blank group, MCS, and miR533). (B) mRNA expression levels of TNF-α and IL-6 in skin samples. (C) Expression of NF-κB and target genes thereof in skin samples detected by qPCR (n=6 mice).

FIG. 13 is a schematic diagram illustrating the effect of pAAV-DMP-miR533 on apoptosis and viability of CT-26 cells. CT-26 cells were transfected with various plasmids and cultured for 24, 48, and 72 h, respectively. (A) Representative images of flow cytometric analysis of cell apoptosis. (B) Cell apoptosis (n=3 wells). (C) Cell viability (n=3 wells). Lipo, cells treated with Lipofectamine 2000. NT, cells transfected with pAAV-DMP-NT; miR533, cells transfected with pAAV-DMP-miR533.

FIG. 14 is a schematic diagram illustrating the effect of pAAV-DMP-miR533 on apoptosis and viability of NIH-3T3 cells. NIH-3T3 cells were induced with or without TNF-α (final concentration of 10 ng/ml) for 1 h before transfection. NIH-3T3 cells were transfected with various plasmids and then cultured for 24, 48, and 72 h, respectively. (A) Representative images of flow cytometric analysis of cell apoptosis. (B) Cell apoptosis (n=3 wells). (C) Cell viability (n=3 wells). NT, cells transfected with pAAV-DMP-NT; miR533, cells transfected with pAAV-DMP-miR533.

FIG. 15 is a schematic diagram illustrating the detection of NF-κB and target gene expression thereof in CT26 and NIH-3T3 cells. Transfection with pAAV-DMP-miR533 for 48 h. NIH-3T3 cells were induced with TNF-α at a final concentration of 10 ng/ml for 1 h before transfection as needed. The expression of NF-κB and target genes thereof in CT26 (A) and NIH-3T3 (B) cells was detected by PCR. Lipo, cells treated with lipo2000. NT, cells transfected with pAAV-NT. miR533, cells transfected with pAAV-DMP-miR533.

FIG. 16 is a schematic diagram illustrating the treatment of arthritis mice with rAAV-DMP-miR533. The CIA mouse model was established by collagen induction and treated by intravenous injection of rAAVs (iv). (A) A schematic diagram of the construction and treatment of the CIA mouse model. PBS, healthy group treated with phosphate-buffered saline. (B) Representative images of the front and back paws of different groups. (C) Visual images of mouse spleen. (D) Spleen weight (n=6 mice). (E) Average weight of mice in each group (n=6 mice). (F) Clinical scores of arthritis severity (n=6 mice). (G) Paw thickness (n=6 mice). (H) Ankle thickness (n=6 mice). (I) Tail width (n=6 mice). (J) ELISA detection of TNF-α and IL-6 levels in serum (n=6 mice). (K) qPCR detection of TNF-α and IL-6 mRNA levels in hind paw samples (n=6 mice). (L) Micro-CT imaging of hind paws and ankle joints. The contour areas show the high-resolution micro-CT images. (M) H&E-stained sections of representative ankle joints. Scale bar: 50 μm. (N) Histopathological scores of ankle joints (n=6 mice). PBS group, normal mice treated with PBS; CIA group, CIA mice treated with PBS; MTX group, CIA mice treated with methotrexate (MTX); NT group, CIA mice treated with rAAV-NT; miR533 group, CIA mice treated with rAAV-DMP-miR533. ns, no significant difference. NT, non-targeted microRNA.

FIG. 17 is a schematic diagram illustrating the treatment of arthritic mice with rAAV-DMP-miR533. A collagen-induced arthritis (CIA) mouse model was established by collagen induction and treated by intravenous (iv) injection of rAAVs. Images of the front and hind paws of all mice in different groups (PBS, CIA, NT, MTX, and miR533) (n=6 mice; PBS, normal mice treated with phosphate-buffered saline (PBS); CIA, CIA mice treated with PBS; MTX, CIA mice treated with methotrexate (MTX); NT, CIA mice treated with rAAV-NT; miR533, CIA mice treated with rAAV-DMP-miR533. ns, not significant. NT, no target.

FIG. 18 is a schematic diagram illustrating the treatment of arthritic mice with rAAV-DMP-miR533. A collagen-induced arthritis (CIA) mouse model was established by collagen induction and treated by intravenous (iv) injection of rAAVs. (A) Representative H&E-stained sections of ankle joints of all mice. Scale bar: 50 μm. (B) qPCR detected the expression of NF-κB and target genes thereof in the hind paws (n=6 mice). PBS, normal mice treated with phosphate-buffered saline (PBS); CIA, CIA mice treated with PBS; MTX, CIA mice treated with methotrexate (MTX); NT, CIA mice treated with rAAV-NT; miR533, CIA mice treated with rAAV-DMP-miR533. ns, not significant. NT, no target.

FIG. 19 is a schematic diagram illustrating the treatment of arthritic mice with rAAV-DMP-miR533. A collagen-induced arthritis (CIA) mouse model was established by collagen induction and treated by intravenous (iv) injection of rAAVs. (A) Representative H&E-stained sections of major organs. Scale bar: 100 μm. (B) Serum biochemical indices of liver (n=6 mice). (C) Serum biochemical indices of kidney (n=6 mice). PBS, normal mice treated with phosphate-buffered saline (PBS); CIA, CIA mice treated with PBS; MTX, CIA mice treated with methotrexate (MTX); NT, CIA mice treated with rAAV-NT; miR533, CIA mice treated with rAAV-DMP-miR533. ns, not significant. NT, no target. ALT, alanine aminotransferase; AST, aspartate aminotransferase; ALP, alkaline phosphatase; BUN, blood urea nitrogen; Cr, creatinine; UA, uric acid.

FIG. 20 is a schematic diagram illustrating the effects of pAAV-DMP-miR533 and pAAV-DMP-miR533-5 on apoptosis and viability of CT-26 and NIH-3T3 cells. Cells were transfected with various plasmids and then cultured for 24, 48, and 72 h, respectively. (A) Representative fluorescence images of OA-stained HL7702 cells. (B) Cell viability (n=3 wells). pAAV-DMP-miR533 contains a single copy of DMP-miR533. pAAV-DMP-miR533-5 contains five copies of DMP-miR533. (C) A schematic diagram of the effects of pAAV-DMP-miR533 and pAAV-DMP-miR533-5 on apoptosis and viability of CT-26 and NIH-3T3 cells.

### DETAILED DESCRIPTION

The present disclosure is further described below with reference to the accompanying drawings and embodiments.

### Example 1

### Vector Construction and Virus Preparation

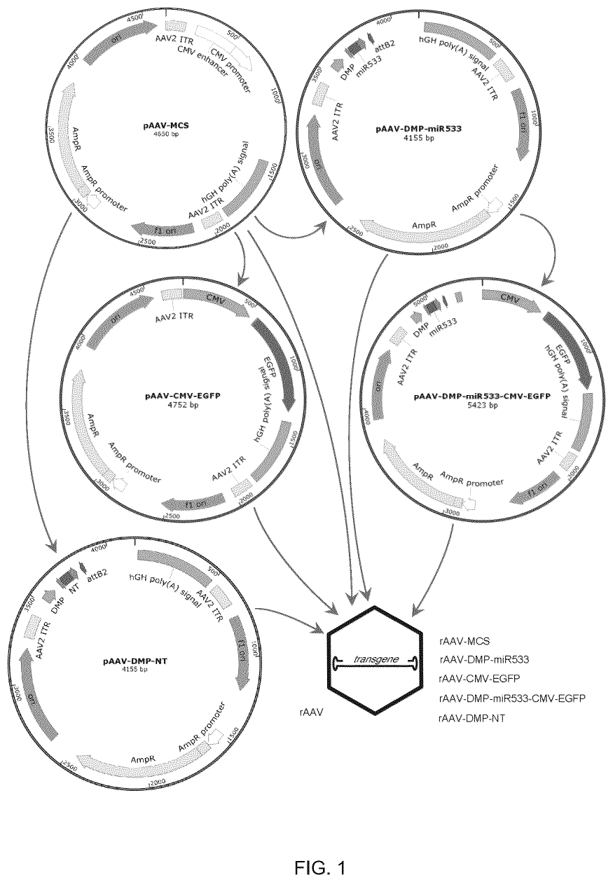

Vector construction: a microRNA sequence targeting human or mouse RELA was designed on the BLOCK-iT™ RNAi Designer website (https://rnaidesigner.thermofisher.com/rnaiexpress/) (Tables 1-2). The DMP-miR533 fragment was amplified from a pDMP-miR533 vector and then the DMP-miR533 fragment was ligated to a pAAV-MCS vector (VPK-410, Stratagene) using Mlul (upstream) and Xbal (downstream) restriction sites to construct pAAV-DMP-miR533 (FIG. 1). A CMV-EGFP fragment was amplified using a pair of primers with the upstream Mlul and downstream EocRI restriction sites from pEGFP-C1 (Clontech), and then the CMV-EGFP fragment was cloned into the pAAV-MCS to obtain a vector pAAV-CMV-EGFP (FIG. 1). The DNA fragments were PCR amplified using Hieff™ PCR Master Mix (With Dye) (Yeasen). The DNA fragments amplified by PCR were purified and recovered by agarose gel electrophoresis and an AxyPrep DNA gel extraction kit (Axygen). The digestion and ligation reactions included appropriate restriction endonucleases (ThermoFisher Scientific) and T4 DNA ligase (ThermoFisher Scientific). The plasmid pAAV-DMP-miR533-CMV-EGFP was obtained by constructing the CMV-EGFP fragment into the pAAV-DMP-miR533 vector (FIG. 1). As a negative control vector, the miR-NT fragment was synthesized according to the sequence of the plasmid pcDNA™ 6.2-GW/EmGFP-miR-Neg (Thermo Fisher Scientific) and the miR-NT fragment was inserted into pDMP-miR to obtain the pDMP-NT plasmid vector. The DMP-NT fragment was copied from the pDMP-NT and inserted into pAAV-MCS to obtain the pAAV-DMP-NT vector (FIG. 1). All plasmids including pAAV-MCS, pAAV-DMP-NT, pAAV-CMV-EGFP, pAAV-DMP-miR533, pAAV-DMP-miR533-CMV-EGFP, pAAV-Helper, and pAAV-RC were transfected into E. coli DH5a (Tiangen) and purified using EndoFree Plasmid Kit (CWBio). All plasmids were verified by DNA sequencing. Oligonucleotides and primers used in this Example were synthesized by Sangon Biotech (Shanghai, China) (Tables 2-3).

TABLE 1
Target sequences of miRNA

| Name | miRNA target (5′-3′) |
|---|---|
| Human RELA | CAAAGATGGGATGAGAAAGGA (SEQ |
| | ID NO. 3) |
| Mouse RELA | TACTCTTGAAGGTCTCATAGG (SEQ |
| | ID NO. 4) |
| NT | GTCTCCACGCGCAGTACATTT (SEQ |
| | ID NO. 5) |

TABLE 2
Oligonucleotide sequences used to construct miRNA expression vectors

| Name | Sequence (5′-3′) |
|---|---|
| Human | TGCTGCAAAGATGGGATGAGAAAGGAGTTTTGGCCACTGACTGACTCC |
| miRELA-F | TTTCTTCCCATCTTTG (SEQ ID NO. 6) |
| Human | CGTTTCTACCCTACTCTTTCCTCAAAACCGGTGACTGACTGAGGAAAG |
| miRELA-R | AAGGGTAGAAACGTCC (SEQ ID NO. 7) |
| Mouse | TGCTGTACTCTTGAAGGTCTCATAGGGTTTTGGCCACTGACTGACCCT |
| miRELA-F | ATGAGCTTCAAGAGTA (SEQ ID NO. 8) |
| Mouse | CCTGTACTCTTGAAGCTCATAGGGTCAGTCAGTGGCCAAAACCCTATG |
| miRELA-R | AGACCTTCAAGAGTAC (SEQ ID NO. 9) |
| miNT-F | TGCTGAAATGTACTGCGCGTGGAGACGTTTTGGCCACTGACTGACGTC |
| | TCCACGCAGTACATTT (SEQ ID NO. 10) |
| miNT-R | CCTGAAATGTACTGCGTGGAGACGTCAGTCAGTGGCCAAAACGTCTCC |
| | ACGCGCAGTACATTTC (SEQ ID NO. 11) |

Cell culture: all cell lines used in the example were from the Cell Resource Center of the Shanghai Institute of Life Sciences, Chinese Academy of Sciences, including HEK-293T (human fetal kidney cells), HT-29 (human colon cancer cells), CT-26 (mouse colon cancer cells), HL7702 (human normal liver cells), and NIH-3T3 (mouse embryonic fibroblasts). HT-29, CT-26, and HL7702 cells were cultured in Roswell Park Memorial Institute (RPMI) 1640 culture medium (Gibco). NIH-3T3 and HEK-293T were cultured in Dulbecco's Modified Eagle Medium (DMEM) (Gibco). All culture media were supplemented with a final content of 10% fetal bovine serum (HyClone), 100 units/mL penicillin (Thermo Fisher), and 100 μg/mL streptomycin (Thermo Fisher). All cells were cultured in a 37° C. humidified incubator containing 5% CO₂.

Virus preparation: HEK-293T cells were seeded at a density of 5×10⁶ cells per flask in a 75 cm² culture flask and cultured for 24 h. Then, two helper plasmids (pAAV-Helper and pAAV-RC; Stratagene) and one pAAV plasmid (pAAV-MCS, pAAV-DMP-NT, pAAV-DMP-miR533, pAAV-DMP-miR533-CMV-EGFP, and pAAV-CMV-EGFP) were co-transfected using Lipofectamine 2000 (Thermo Fisher) according to the instructions. After the cells were cultured for another 72 h, the cells and the culture medium were collected and stored at −80° C. overnight. The cells and the culture medium were then incubated in a 37° C. water bath for 2 h. The entire freeze-thawing process was repeated three times. Pure chloroform was added to a cell lysate after freeze-thawing at a volume ratio of 1:10 to obtain a mixture, and the mixture was vigorously shaken at 37° C. for 1 h. After shaking, NaCl was added to the mixture to a final concentration of 1 mole and shaken until the NaCl was dissolved to obtain a solution. The solution was centrifuged at 15,000 rpm for 15 min at 4° C., and a supernatant was collected. PEG8000 was added to a final concentration of 10% (w/v) and shaken until the PEG8000 was dissolved. The reaction was centrifuged at 15,000 rpm for 15 min at 4° C., the supernatant was discarded, and a precipitate was dissolved in PBS. DNase and RNase were then added to the dissolved precipitate to a final nuclease concentration of 1 μg/mL. The reaction was incubated at room temperature for 30 min. Finally, an incubated agent was extracted once with chloroform (1:1 volume), and an aqueous phase containing the purified virus was transferred to a new test tube. A titer of AAV was determined by using primers AAV-F/R and qPCR detection (Table 3). After the virus was quantified, the virus was segmented and stored at −80° C. for later use. The obtained viruses were named as rAAV-MCS, rAAV-DMP-NT, rAAV-DMP-miR533, rAAV-DMP-miR533-CMV-EGFP, and rAAV-CMV-EGFP, respectively.

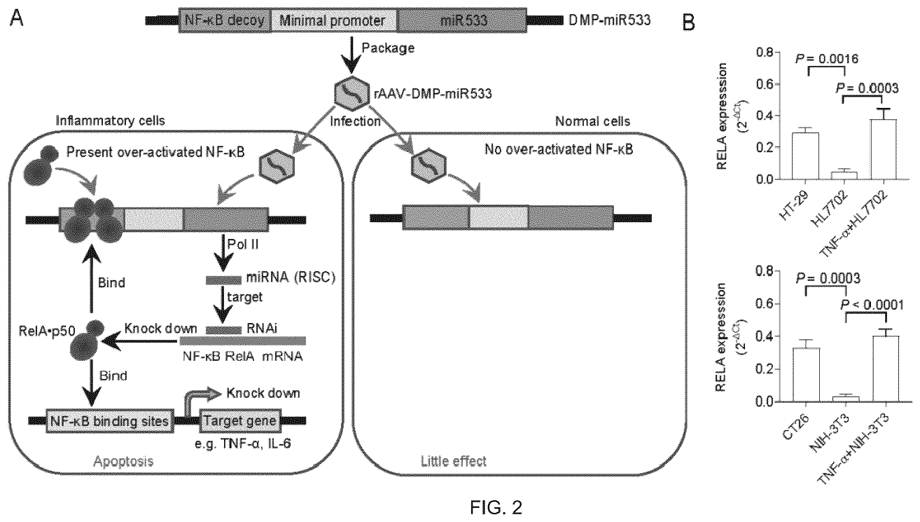

NF-κB is widely over-activated in inflammatory cells. In order to inhibit NF-κB activity, this Example designs an rAAV named rAAV-DMP-miR533 (FIG. 2A), in which DMP is a promoter composed of a NF-κB decoy and a minimal promoter, and miR533 encodes an artificial microRNA targeting NF-κB RELA. When DMP-miR533 is transfected into inflammatory cells such as human colon cancer cells (HT-29), mouse colon cancer cells (CT-26), TNFα-induced human normal liver cells (HL7702), and mouse embryonic fibroblasts (NIH-3T3) (FIG. 2B), the DMP binds to the over-activated NF-κB, thereby activating the transcription of miR533. When NF-κB is inhibited by miR533 interference, the expression of inflammatory cytokines (NF-κB target genes), such as TNF-α and IL-6, is downregulated. Therefore, the inhibition of NF-κB can further lead to apoptosis of inflammatory cells. In contrast, in normal cells such as human normal hepatocytes (HL7702) and mouse embryonic fibroblasts (NIH-3T3) (FIG. 2B), the DMP-miR533 is unable to function due to the lack of NF-κB activity (FIG. 2A). In order to fully evaluate the anti-inflammatory effects of the DMP-miR533 in cells and animals, miR533 targeting human and mouse NF-κB RELA transcripts was designed and prepared, respectively (Table 1). Human cells were treated with DMP-miR533 targeting human NF-κB RELA, and mouse cells and mice were treated with DMP-miR533 targeting mouse NF-κB RELA.

In this Example, a serotype AAV2 was used as a recombinant adeno-associated virus vector. In this Example, the functional DNA fragment DMP-miR was inserted into the pAAV-MCS vector (VPK-410, Stratagene), and two helper plasmids pAAV-Helper and pAAV-RC were used to co-transform 293T cells to prepare recombinant adeno-associated viruses. The recombinant adeno-associated virus packaged and prepared by the above three-plasmid system was a serotype AAV2 recombinant adeno-associated virus because the helper plasmid pAAV-RC contains the Rep and Cap genes of AAV2. AAV2 has a relatively wide range of tissue infection ability, such that the constructed rAAV-DMP-miR533 can be used universally to treat inflammations in different tissues and organs, such as inflammatory colitis in intestinal tissues, inflammatory psoriasis in skin tissues, and inflammatory arthritis in joint tissues treated by the present disclosure. If a serotype of AAV with a significant tissue infection bias is used, such as AAV9 that is biased towards neural tissue, when the constructed rAAV is used to treat inflammation of different tissues and organs, it is necessary to construct different serotypes of rAAV-DMP-miR533, which is a cumbersome and costly process. In addition, AAV2 is also one of the natural adeno-associated viruses. After a long period of coexistence and evolution with humans, the human body has extremely low immunogenicity to this AAV, making it safer to use. In addition, the patent for AAV2 has expired, and there are no patent restrictions in the preparation of therapeutic agents, which is conducive to promotion and application.

### Example 2

### Inflammatory Cell Treatment

Cells (HT-29, CT-26, HL7702, and NIH-3T3) (1×10⁵) were seeded into a 24-well plate and cultured overnight at 37° C. with 5% CO₂. Then, various pAAV plasmids (500 ng/well) prepared in the Example 1 were transfected into cells using Lipofectamine2000 (Thermo Fisher) according to the instructions. After transfection, the cells were cultured for 24 h, 48 h, and 72 h, respectively. If necessary, normal cells were induced with TNF-α (Sigma-Aldrich) at a final concentration of 10 ng/ml for 1 h before transfection. Subsequently, the cells were stained with acridine orange (Solarbio) according to the instructions, in which live cells showed a uniform green color. The cells were imaged with a fluorescence microscope (IX51, Olympus) and counted using Image-Pro Plus software.

Cell viability assay: cell viability was measured and analyzed using Cell Counting Kit-8 (CCK-8, Yeasen). Cells (HT-29, CT-26, HL7702, and NIH-3T3) (5×10³) were seeded into a 96-well plate and cultured overnight at 37° C. with 5% CO₂. Then, various pAAV plasmids prepared in the Example 1 (200 ng/well) were transfected into the cells using Lipofectamine 2000 (Thermo Fisher). After transfection, the cells were cultured for 24 h, 48 h, and 72 h, respectively. If necessary, normal cells were induced with TNF-α (Sigma-Aldrich) at a final concentration of 10 ng/ml for 1 h before transfection. Finally, CCK-8 agent (10 μL/well) was added to the cells and incubated for 1 h to obtain a solution, and the absorbance of the solution was measured at 450 nm using a microplate reader (BioTek).

Apoptosis detection: cells (HT-29, CT-26, HL7702, and NIH-3T3) (5×10⁵) were seeded into a 6-well plate and cultured overnight at 37° C. with 5% CO₂. Then, various pAAV plasmids prepared in the Example 1 (4 μg/well) were transfected into the cells using Lipofectamine 2000 (Thermo Fisher). After transfection, the cells were cultured for 24 h, 48 h, and 72 h, respectively. If necessary, normal cells were induced with TNF-α (Sigma-Aldrich) at a final concentration of 10 ng/ml for 1 h before transfection. Then, quantification of cell apoptosis was analyzed by using AnnexinV-FITC/PI apoptosis detection kit (Vazyme) and flow cytometry (Calibur, BD, USA) according to the manufacturer's instructions.

Cell EGFP fluorescence detection: cells (HT-29, CT-26, HL7702, and NIH-3T3) (5×10³) were seeded into a 96-well plate and cultured overnight at 37° C. with 5% CO₂. Then the cells were infected with various rAAVs prepared in the Example 1 (5×10⁷ vg/well). After infection, the cells were cultured for 48 h. If necessary, normal cells were induced with TNF-α (Sigma-Aldrich) at a final concentration of 10 ng/ml for 1 h before infection. EGFP fluorescence was imaged using a fluorescence microscope (IX51, Olympus) and quantitatively analyzed by flow cytometry (Calibur, BD, USA). Cell apoptosis was detected using the AnnexinV-FITC/PI apoptosis detection kit and flow cytometry.

Quantitative PCR (qPCR) detection of gene expression: total RNA was isolated from cultured cells and mouse tissues using TRIzol™ (Invitrogen) according to the protocol provided by the instructions. Then complementary DNA (cDNA) was generated using a PrimeScript™ RT Kit with gDNA Eraser (Takara). Fast SYBR Green Master Mix (Roche) was added to the samples, and the expression of target genes on cDNA was detected by quantitative PCR (qPCR) on an ABI StepOne Plus instrument (Applied Biosystems). Three technical replicates were performed for each sample. Relative mRNA transcript levels were calculated as 2^{-ΔCt} or 2^{-ΔΔCt}, where ΔCt=Ct_{target}−Ct_{GADPH}; ΔΔCt=ΔCtₜᵣₑₐₜₘₑₙₜ−ΔCt_{control}. 2^{-ΔΔCt} was also defined as relative quantification (RQ). The specificity of all qPCR primers (Table 3) was verified using melting curve analysis.

TABLE 3
Primer sequences used for qPCR

| | Primer sequence | | Primer sequence |
|---|---|---|---|
| Name | (5′-3′) | Name | (5′-3′) |
| Human | CCTGGAGCAGGCTATC | Human | ATGGGATGAGAAAGGA |
| RELA-F | AGTC (SEQ ID NO. 12) | RELA-R | CAGG (SEQ ID NO. 13) |
| Mouse | AGGCTTCTGGGCCTTA | Mouse | TGCTTCTCTCGCCAGG |
| RELA-F | TGTG (SEQ ID NO. 14) | RELA-R | AATAC (SEQ ID NO. 15) |
| Human | ATTTGGTCGTATTGGG | Human | CTCGCTCCTGGAAGAT |
| GAPDH-F | CG (SEQ ID NO. 16) | GAPDH-R | GG (SEQ ID NO. 17) |
| Mouse | AGGTCGGTGTGAACG | Mouse | TGTAGACCATGTAGTT |
| GAPDH-F | GATTTG (SEQ ID | GAPDH-R | GAGGTCA (SEQ ID |
| | NO. 18) | | NO. 19) |
| Human | GAACACCGAGTGCCA | Human | GCTAAGGCTGTTGTTT |
| BCL3-F | AGAAACC (SEQ ID | BCL3-R | TCCACGG (SEQ ID |
| | NO. 20) | | NO. 21) |
| Human | AGGTGACGCTGAATG | Human | GCTCGGGCAATGGGT |
| CD54-F | GG (SEQ ID NO. 22) | CD54-R | T (SEQ ID NO. 23) |
| Human | TCCACTCCATCCTGAA | Human | CAAGGACACCAAAAG |
| NFKBIA-F | GGCTAC (SEQ ID | NFKBIA-R | CTCCACG (SEQ ID |
| | NO. 24) | | NO. 25) |
| Human | GCAGCACTACTTCTTG | Human | TCTGCTCCTGAGCATT |
| NFKB1-F | ACCACC (SEQ ID | NFKB1-R | GACGTC (SEQ ID |
| | NO. 26) | | NO. 27) |
| Human | GGCAGACCAGTGTCAT | Human | CAGCAGAAAGCTCAC |
| NFKB2-F | TGAGCA (SEQ ID | NFKB2-R | CACACTC (SEQ ID |
| | NO. 28) | | NO. 29) |
| Human | AGAATCACCAGCAGCA | Human | TCCTGAACCCACTTCT |
| CCL2-F | AGTGTCC (SEQ ID | CCL2-R | GCTTGG (SEQ ID |
| | NO. 30) | | NO. 31) |
| Human | AGCTTGCCTCAATCCT | Human | TCCTTCAGGAACAGCC |
| CXCL1-F | GCATCC (SEQ ID | CXCL1-R | ACCAGT (SEQ ID |
| | NO. 32) | | NO. 33) |
| Human | CGGTGAAACTCTGGCT | Human | GCAAACCGTAGATGCT |
| PTGS2-F | AGACAG (SEQ ID | PTGS2-R | CAGGGA (SEQ ID |
| | NO. 34) | | NO. 35) |
| Human | GCCACTACTGTGCCTT | Human | CCCTCAGAGAATCGCC |
| MMP9-F | TGAGTC (SEQ ID | MMP9-R | AGTACT (SEQ ID |
| | NO. 36) | | NO. 37) |
| Mouse | AGCAGTCGTCTCAGCT | Mouse | AGGCAGGTGTAGATGT |
| BCL3-F | CCAATG (SEQ ID | BCL3-R | TGTGGG (SEQ ID |
| | NO. 38) | | NO. 39) |
| Mouse | GTGATGCTCAGGTATC | Mouse | CACAGTTCTCAAAGCA |
| CD54-F | CATCCA (SEQ ID | CD54-R | CAGCG (SEQ ID NO. 41) |
| | NO. 40) | | |
| Mouse | GCCAGGAATTGCTGA | Mouse | GTCTGCGTCAAGACTG |
| NFKBIA-F | GGCACTT (SEQ ID | NFKBIA-R | CTACAC (SEQ ID |
| | NO. 42) | | NO. 43) |
| Mouse | GCTGCCAAAGAAGGA | Mouse | GGCAGGCTATTGCTCA |
| NFKB1-F | CACGACA (SEQ ID | NFKB1-R | TCACAG (SEQ ID |
| | NO. 44) | | NO. 45) |
| Mouse | TGCTGATGGCACAGG | Mouse | GTTGATGACGCCGAG |
| NFKB2-F | ACGAGAA (SEQ ID | NFKB2-R | GTACTGA (SEQ ID |
| | NO. 46) | | NO. 47) |
| Mouse | GCTACAAGAGGATCAC | Mouse | GTCTGGACCCATTCCT |
| CCL2-F | CAGCAG (SEQ ID | CCL2-R | TCTTGG (SEQ ID |
| | NO. 48) | | NO. 49) |
| Mouse | TCCAGAGCTTGAAGGT | Mouse | AACCAAGGGAGCTTCA |
| CXCL1-F | GTTGCC (SEQ ID | CXCL1-R | GGGTCA (SEQ ID |
| | NO. 50) | | NO. 51) |
| Mouse | GCGACATACTCAAGCA | Mouse | AGTGGTAACCGCTCAG |
| PTGS2-F | GGAGCA (SEQ ID | PTGS2-R | GTGTTG (SEQ ID |
| | NO. 52) | | NO. 53) |
| Mouse | GCTGACTACGATAAGG | Mouse | TAGTGGTGCAGGCAG |
| MMP9-F | ACGGCA (SEQ ID | MMP9-R | AGTAGGA (SEQ ID |
| | NO. 54) | | NO. 55) |
| Mouse | CCCTCACACTCAGATC | Mouse | GCTACGACGTGGGCTA |
| TNF-α-F | ATCTTCT (SEQ ID | TNF-α-R | CAG (SEQ ID NO. 57) |
| | NO. 56) | | |
| Mouse | TAGTCCTTCCTACCCC | Mouse | TTGGTCCTTAGCCACT |
| IL-6-F | AATTTCC (SEQ ID | IL-6-R | CCTTC (SEQ ID NO. 59) |
| | NO. 58) | | |
| AAV-F | TGCATGACCAGGCTCA | AAV-R | GACAGGGAAGGGAGC |
| | GCTA (SEQ ID NO. 60) | | AGTG (SEQ ID NO. 61) |

Statistical analysis of data: all data were expressed as mean±standard deviation (SD), and statistical analysis and graph drawing were performed by GraphPad Prism 8.0 software. Statistical differences between two groups were determined using a two-tailed Student's T test. Depending on the data, three or more groups were statistically analyzed by using one-way or two-way analysis of variance (ANOVA) with Tukey's or Sidak's multiple comparisons. Differences with p<0.05 were considered statistically significant.

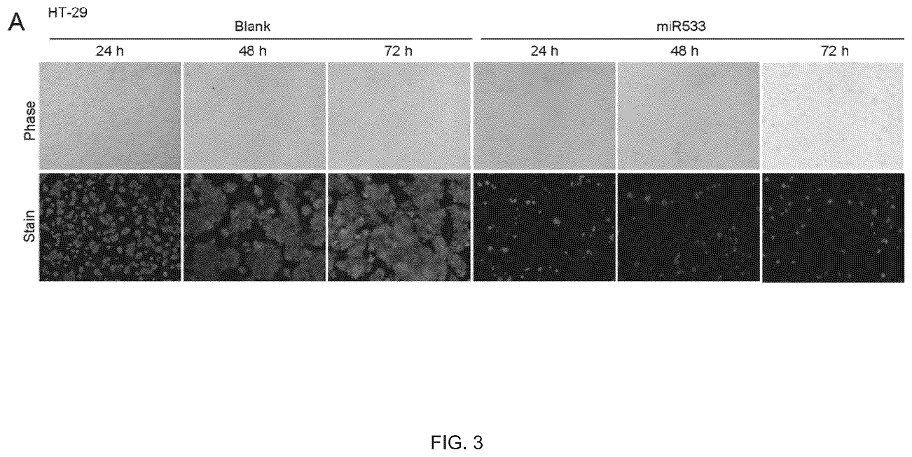

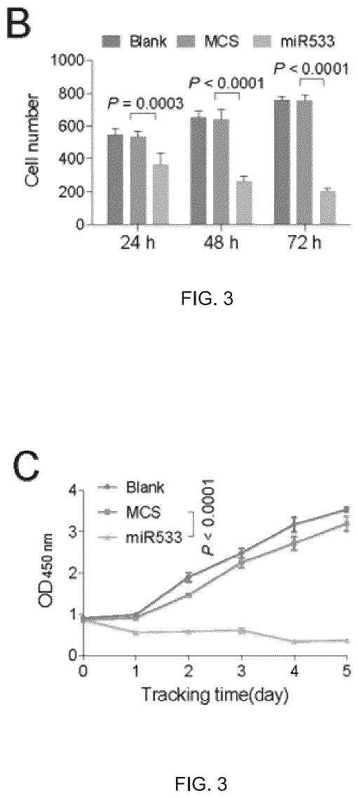

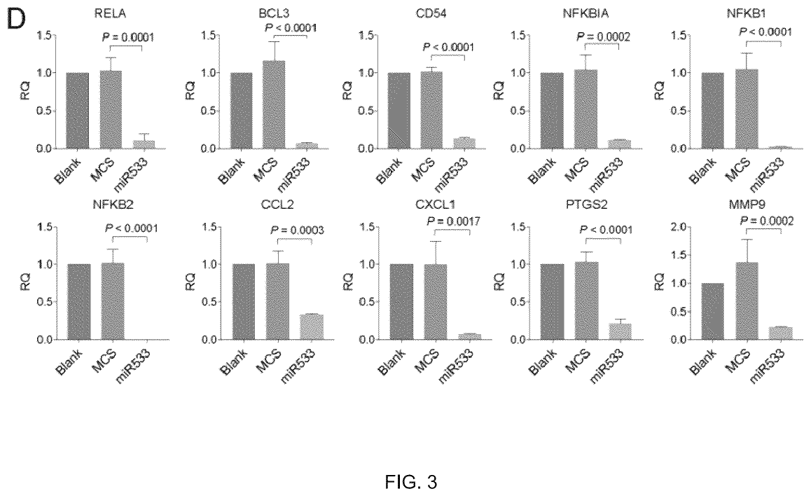

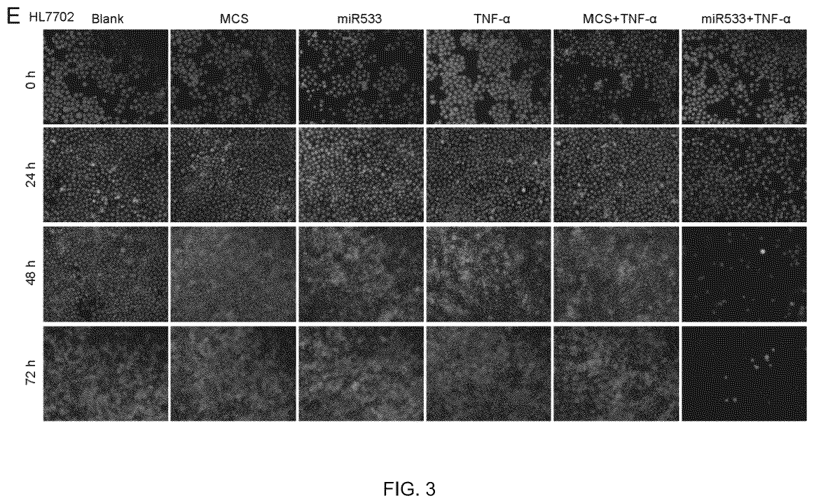

Results: in order to evaluate the feasibility of the DMP-miR533 system in killing inflammatory cells, human colon cancer cells (HT-29) with over-activated NF-κB were first selected as experimental subjects and transfected with pAAV-DMP-miR533 for 24-72 h. Acridine orange (AO) staining of the cells showed that pAAV-DMP-miR533 had significant cytotoxicity to HT-29 cells (FIGS. 3A-3B). The cell growth curve also showed that the growth of HT-29 cells was significantly inhibited by pAAV-DMP-miR533 (FIG. 3C). Meanwhile, pAAV-MCS (empty virus vector) had no effect on cell viability and growth (FIGS. 3B and 3C). To further verify the in vitro interference efficiency of the pAAV-DMP-miR533, the expression of NF-κB RELA and target genes thereof was detected by qPCR. The results showed that the expression of these genes was significantly inhibited by the pAAV-DMP-miR533, but there was no change in the Lipofectamine (blank group) and pAAV-MCS groups (FIG. 3D). These data indicate that the pAAV-DMP-miR533 can significantly inhibit the growth of cells with over-activated NF-κB by knocking down the expression of NF-κB and the target genes thereof.

To further explore whether the pAAV-DMP-miR533 has an effect on inflammation, human normal hepatocytes (HL7702) were induced with TNF-α, a known NF-κB inducer, to construct a cell inflammatory model. Meanwhile, HL7702 cells that were not induced with TNF-α were used as a control. AO staining of the cells showed that the pAAV-DMP-miR533 had no significant effect on normal HL7702 cells, but after TNF-α induction, the count of HL7702 cells was greatly reduced (FIG. 3E). These results indicate that the pAAV-DMP-miR533 can cause inflammatory cell death, but has no obvious effect on non-inflammatory cells.

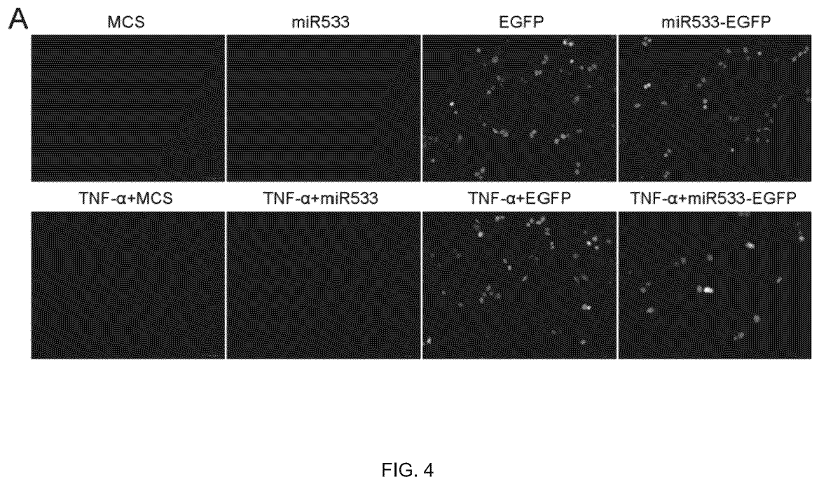

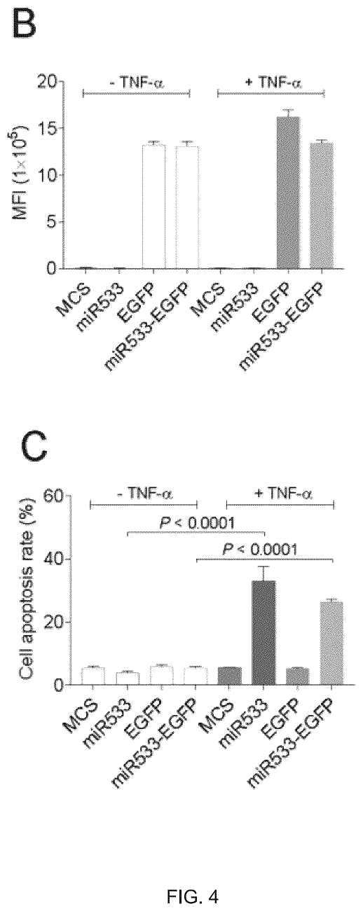

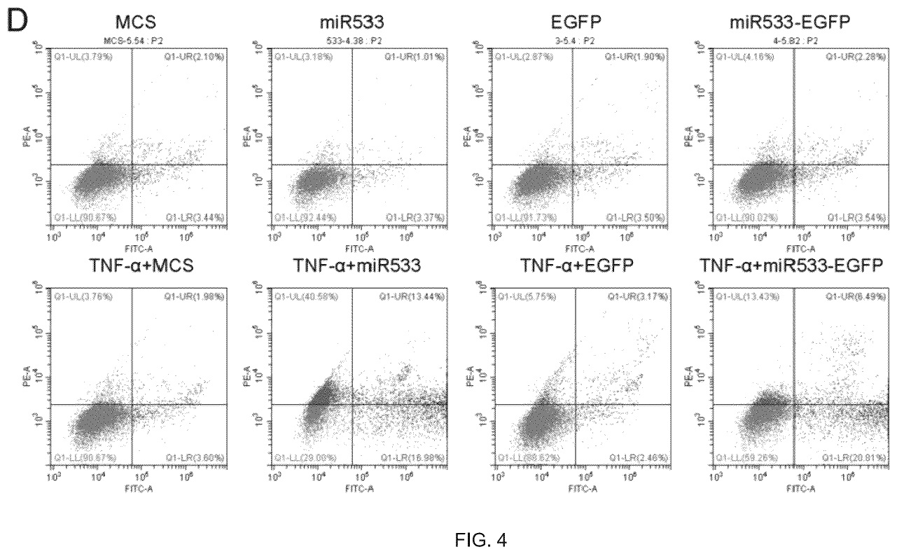

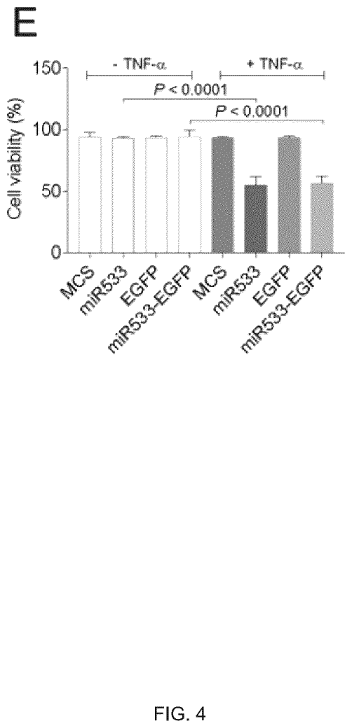

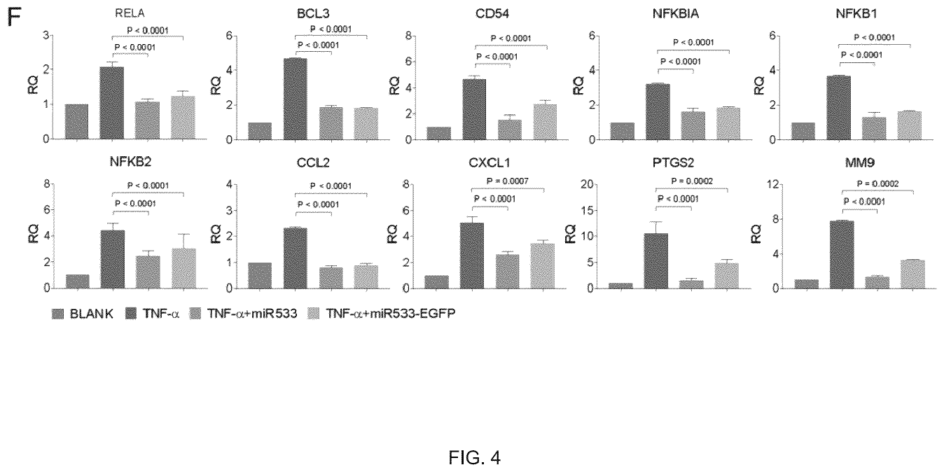

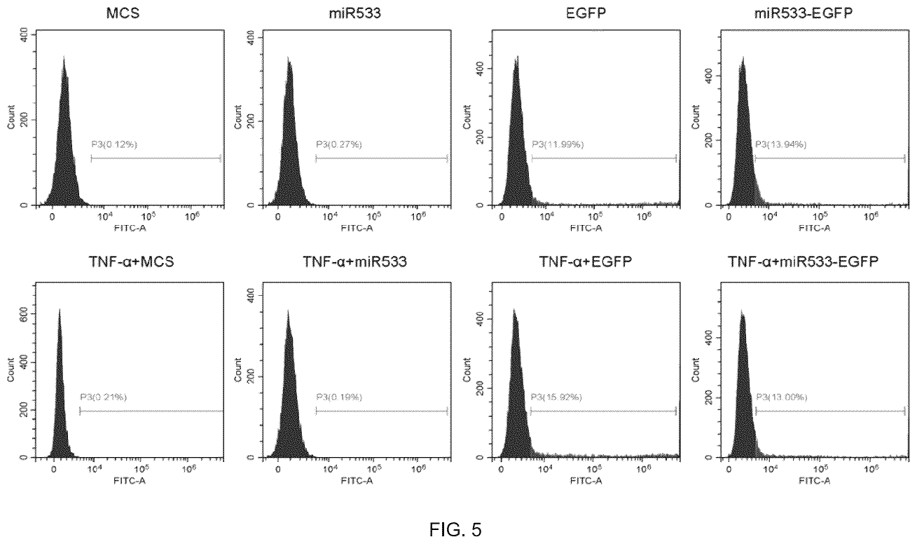

For in vivo application, different DNA fragments were packaged into adeno-associated virus (AAV) to construct rAAV-MCS, rAAV-CMV-EGFP, rAAV-DMP-miR533, and rAAV-DMP-miR533-CMV-EGFP (FIG. 1). Meanwhile, the CMV-EGFP fragment was inserted into the rAAV-DMP-miR533 to monitor the infection of rAAV-DMP-miR533 to the cells. Empty virus rAAV-MCS containing the CMV promoter and no target gene was used as a negative control. First, the packaged viruses were used to infect TNF-α-treated and untreated HL7702 cells, respectively. The expression of EGFP and cell apoptosis were analyzed by flow cytometry. The results showed that EGFP had similar expression levels in rAAV-DMP-miR533-CMV-EGFP-infected HL7702 cells and rAAV-CMV-EGFP-infected cells (FIGS. 4A-4B; FIG. 5), indicating that the prepared virus can effectively infect cells. In addition, both the rAAV-DMP-miR533 and the rAAV-DMP-miR533-CMV-EGFP induced significant apoptosis in HL7702 cells induced by TNF-α. However, the same infection had little effect on normal HL7702 cells (FIGS. 4C-4D). The cell viability assay further showed that only the infection of the rAAV-DMP-miR533 and the rAAV-DMP-miR533-CMV-EGFP could lead to a significant decrease in TNF-α-induced HL7702 cell viability (FIG. 4E).

To further verify the principle of apoptosis of inflammatory cells infected by rAAV-DMP-miR533, the expression of NF-κB RELA and target genes thereof in HL7702 cells was detected by qPCR. The results showed that TNF-α significantly induced the expression of NF-κB RELA and target genes thereof (FIG. 4F). However, the infection of the rAAV-DMP-miR533 and the rAAV-DMP-miR533-CMV-EGFP could reverse this phenomenon (FIG. 4F). In conclusion, the DMP-miR533 can inhibit the expression of NF-κB RELA and further lead to apoptosis of inflammatory cells and decreased vitality, indicating that the rAAV-DMP-miR533 has a good anti-inflammatory effect in vitro.

### Example 3

### Construction and Treatment of Colitis Model

BALB/c mice purchased from Cavens (China) were randomly divided into 4 groups (n=6), including blank group, dextran sulfate sodium (DSS), DSS+MCS, and DSS+miR533 groups. The mice in the blank group drank water, while the mice in the other three groups drank water containing 3% dextran sulfate sodium (DSS) (MW=36000-50000) (MP). On the 3rd and 5th days after drinking water containing 3% DSS, the mice in the DSS+MCS and DSS+miR533 groups were intravenously injected with 100 μL of 1×1010 vg/mL rAAV-MCS and rAAV-DMP-miR533, respectively. Body weight was measured every day. On the 8th day, the mice were killed, and the colon from the anus to the ileocecal region was isolated and blood was collected. At the same time, the colon length of each mouse was measured. Colon tissues were used for preparation of paraffin sections and gene expression detection. The sections were stained with hematoxylin-eosin (H&E), imaged, and scored. The pathological scoring of colon tissue was performed blindly by other technicians unrelated to the project according to four levels: 0 point, no obvious pathological changes; 1 point, focal inflammatory cell infiltration; 2 points, extensive inflammatory cell infiltration; 3 points, diffuse inflammatory cell infiltration; 4 points, inflammatory cell infiltration, tissue degeneration and necrosis, and hyperplasia of fibrous connective tissue.

Determination of serum TNF-α and IL-6 levels: according to the ELISA instructions, the levels of TNF-α and IL-6 in serum were determined using TNF-α (ab208348, abcam) and IL-6 ELISA kits (ab222503, abcam).

Preparation of tissue sections and hematoxylin and eosin (H&E) staining: mouse tissues including heart, liver, spleen, lung, and kidney were dissected, embedded in paraffin, sectioned, and stained with hematoxylin and eosin (H&E) in sequence. In short, the tissues were first cut into blocks and then fixed in 4% paraformaldehyde solution (Sangon Biotech, China) at room temperature overnight. Then the fixed specimens were removed and decalcified, embedded in paraffin, sectioned, and stained with hematoxylin staining solution (C0107, Beyotime), and eosin staining solution (C0109, Beyotime) in sequence. Finally, the prepared slides were imaged and photographed using a microscope (IX51, Olympus). Histopathological scoring was performed blindly by other unrelated researchers.

QPCR detection of gene expression: same as Example 2.

Data statistical analysis: same as Example 2.

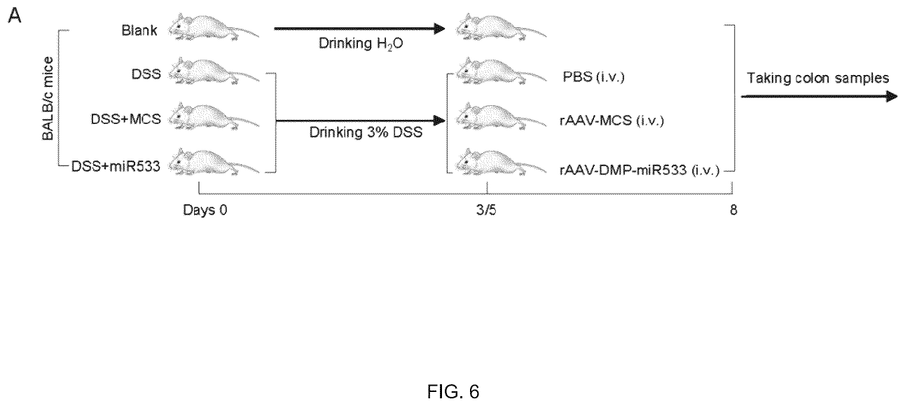

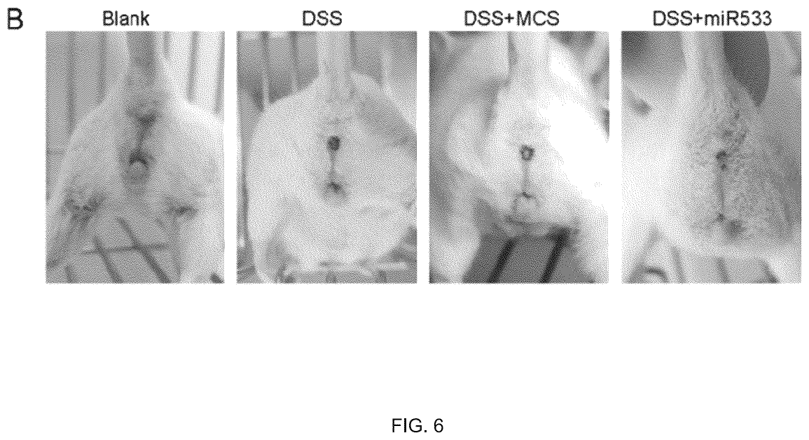

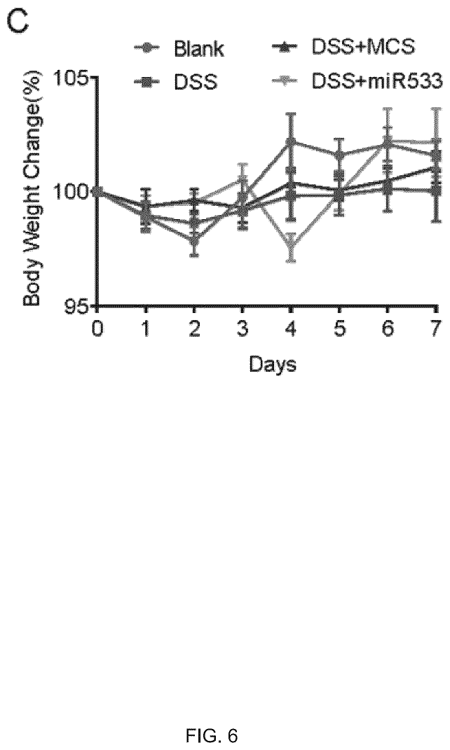

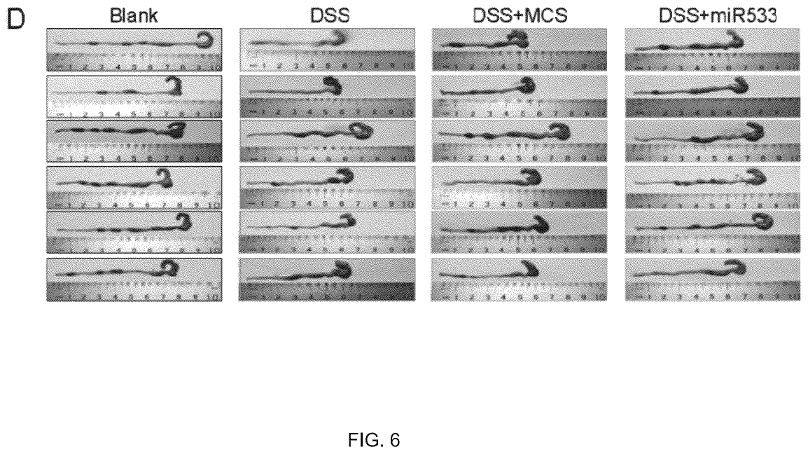

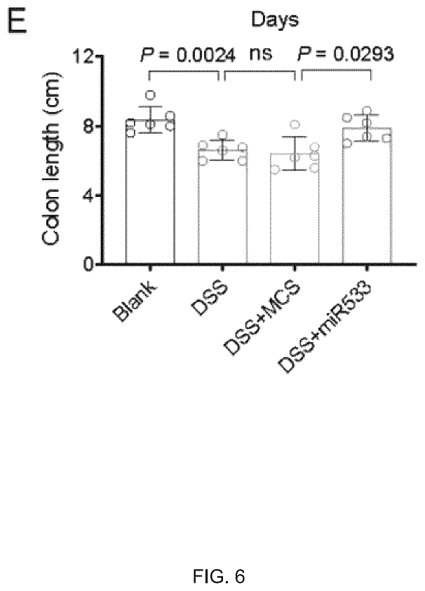

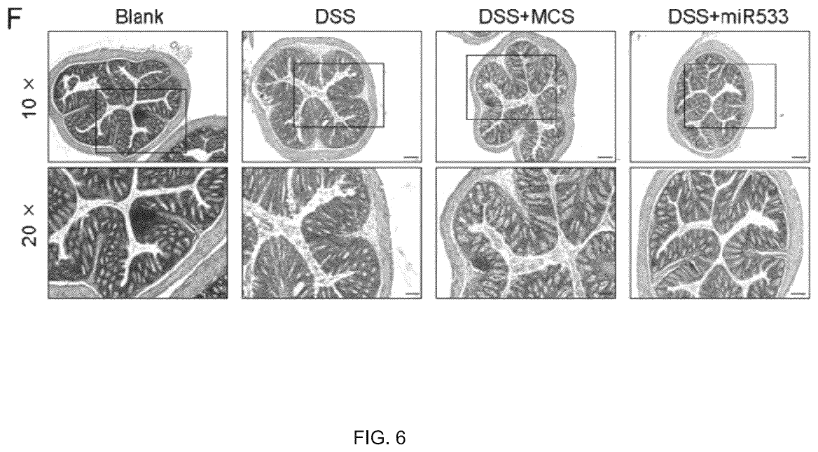

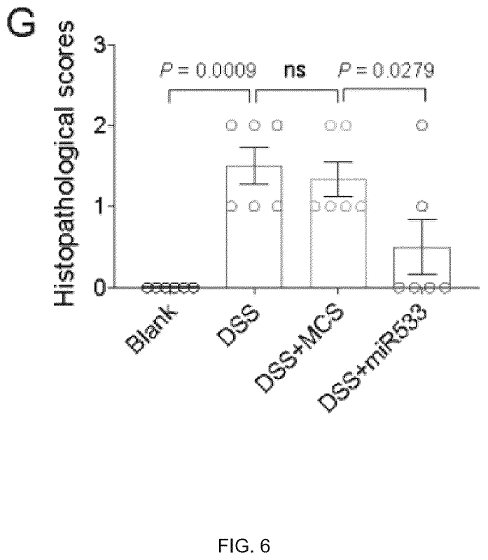

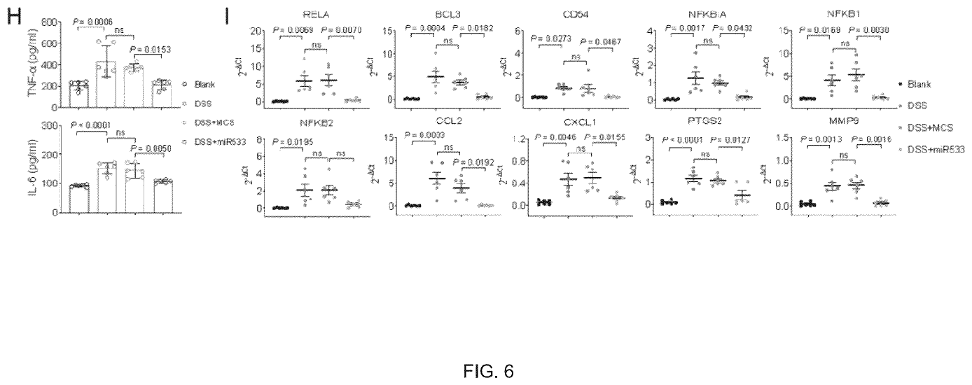

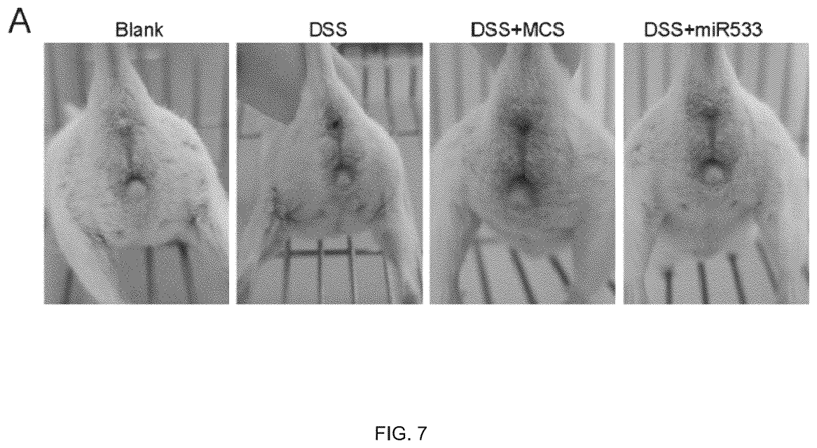

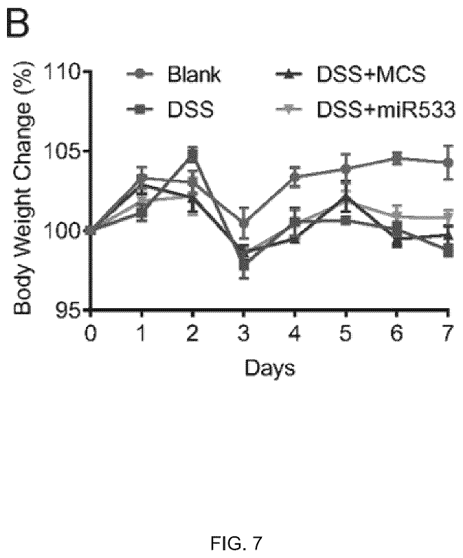

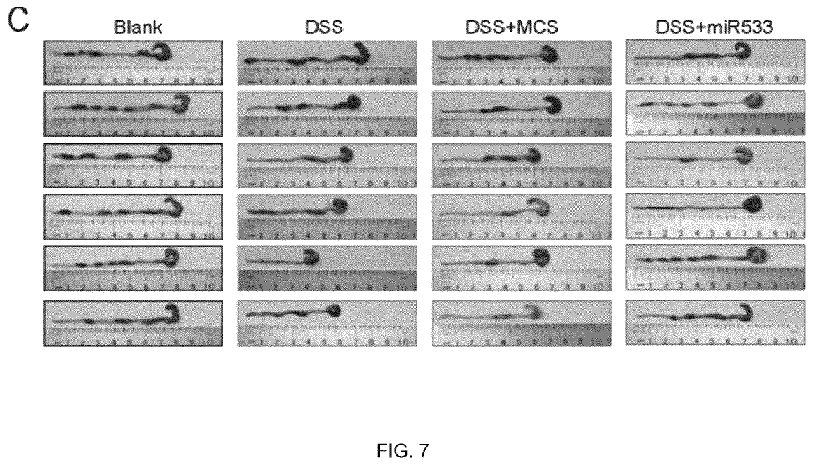

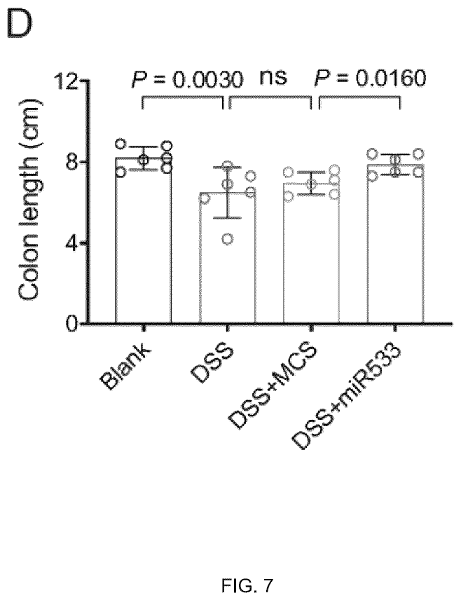

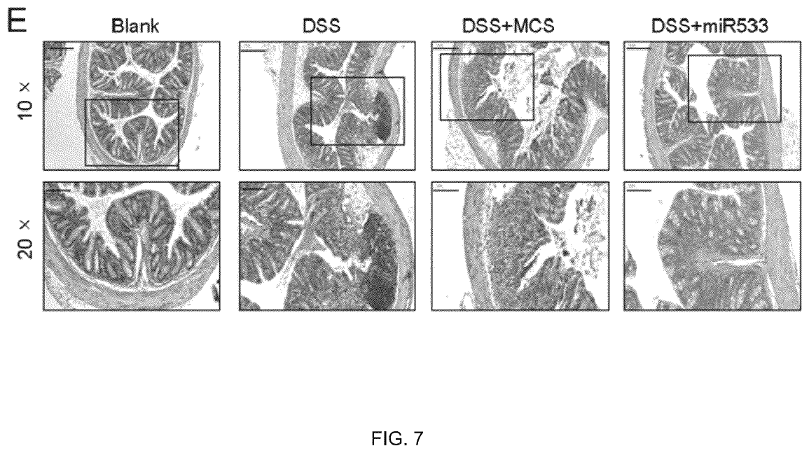

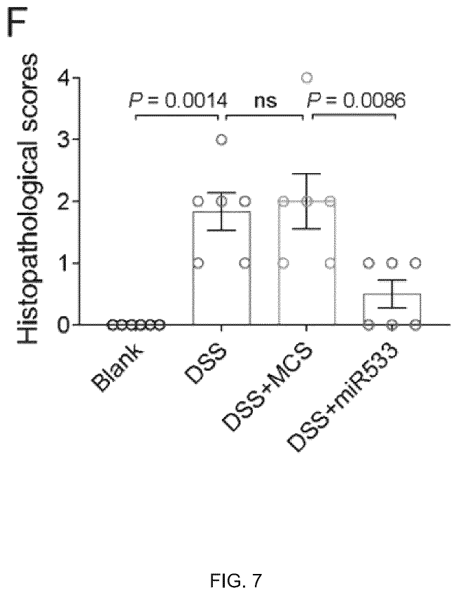

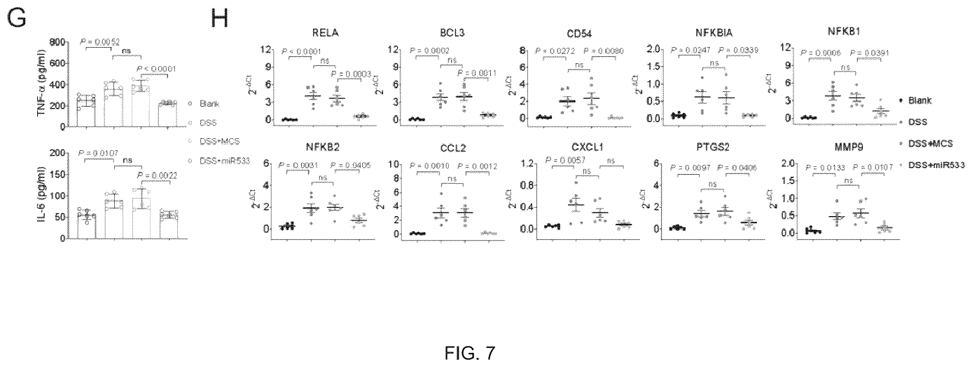

Results: to evaluate the in vivo anti-inflammatory effect of DMP-miR533, dextran sulfate sodium (DSS) was used to induce the construction of a mouse colitis model (FIG. 6A). As shown in FIG. 6B, the mental state and stool status of the four groups of mice were observed every day, and it was found that the mice in the blank group were normal, with hard and bloodless feces. However, the DSS-induced mice gradually became dull and inactive. On the 3rd day after drinking 3% DSS, the feces changed from normal to soft, and on the 5th day, the feces were obviously bloody, and on the 7th day, the anus was still bleeding. These symptoms indicated that DSS-induced acute colitis in mice was successfully modeled. The mice were then treated with various agents. The symptoms of DSS-induced mice treated with rAAV-MCS were similar to those of DSS-induced mice treated with PBS. The feces of both groups of mice became soft and bloody. The fecal status of DSS-induced mice treated with rAAV-miR533 was significantly improved and anal bleeding was significantly reduced. Dynamic measurement of body weight showed that acute colitis mice treated with rAAV-MCS and PBS lost weight, while acute colitis mice treated with the rAAV-DMP-miR533 gained weight (FIG. 6C). The results of the measurement the mouse colon length showed that DSS induction led to a shortening of the mouse colon length (FIGS. 6D and 6E). Only after treatment with rAAV-DMP-miR533 could the length of the mouse colon be restored (FIGS. 6D and 6E). H&E staining sections of colon tissue further showed that after DSS induction, the mouse colon had obvious inflammatory pathological damage, such as the disappearance of mucosal colon crypts, loss of goblet cells, cell degeneration, obvious dense lymphocyte infiltration, and obvious infiltration of neutrophils and plasma cells. However, the treatment with the rAAV-DMP-miR533 reversed these damages, such as that the mouse colon had a relatively intact mucosal layer structure, obvious crypt structure, and very few neutrophil and plasma cell infiltration (FIGS. 6F and 6G). The quantitative results of typical pro-inflammatory factors in serum showed that DSS could induce a significant increase in TNF-α and IL-6 in serum (FIG. 6H). The rAAV-DMP-miR533 significantly reduced the levels of TNF-α and IL-6 (FIG. 6H). In addition, DSS also caused over-activation of NF-κB RELA and target genes thereof (FIG. 6I). Similarly, the treatment with the rAAV-DMP-miR533 significantly downregulated the expression of these genes (FIG. 6I). In addition, another independent biological duplication was carried out for the above animal experiments and similar results were obtained (FIG. 7). In summary, these data fully demonstrate that the rAAV-DMP-miR533 has a significant in vivo anti-inflammatory effect in mice with DSS-induced acute colitis.

### Example 4

### Construction and Treatment of Psoriasis Model

Two animal experiments were conducted on male BALB/c (8 weeks; Cavens, China) psoriasis model mice. The mice were first shaved on their backs (shaved area of approximately 2.5 cm×2.5 cm) and then randomly divided into three groups (n=6), including a blank group, an MCS group, and an miR533 group. The mice in the blank group were treated with petrolatum cream. The mice in the MCS and miR533 groups were treated with 5% w/w imiquimod (IMQ) (Sichuan Mingxin Pharmaceutical, China) on the shaved area on the backs every day at a dose of 62.5 mg/mouse.

In the first animal experiment, 3 mice (n=3) were killed in each of the blank group and MCS group 6 days after IMQ administration, and skin and blood samples were collected. The remaining mice in the MCS group (n=3) and the miR533 group (n=6) were intravenously injected with 100 μL 1×10¹⁰ vg/mL rAAV-MCS and rAAV-DMP-miR533, respectively. The mice in the MCS and miR533 groups continued to be applied with 5% w/w IMQ every day. The mice in the blank group continued to be treated with petrolatum cream. After 6 days, all mice were killed and skin and blood samples were collected. Then RT-qPCR and ELISA kits were used to detect the expression levels of TNF-α and IL-6 in skin and serum samples, respectively. Meanwhile, subcutaneous administration (i.h.) and topical (ad us. ext.) treatment of rAAV-DMP-miR533 were attempted on another psoriasis model mouse (n=1). The psoriasis model mouse was treated with 100 μL 1×10¹⁰ vg/mL rAAV-DMP-miR533 subcutaneously or applied daily for 6 days, after which the mice were skilled and sample collection and testing were the same as in the previous intravenous injection experiment.

In the second animal experiment after 5% w/w IMQ modeling, 100 μL of 1×10¹⁰ vg/mL rAAV-MCS and rAAV-DMP-miR533 were applied to the shaved areas of the backs of mice in the MCS group and the miR533 group (n=6) for 6 days. Similarly, the mice in the MCS group and the miR533 group continued to be applied with 5% w/w IMQ every day. The blank group was applied with petrolatum cream until euthanasia. All mice were killed and photographed on 12th day, and dorsal skin and blood samples were collected. The body weight and psoriasis area and severity index (PASI) of mice were monitored and recorded every day. The erythema, scaling, and thickness on the skin of each mouse were scored independently from 0 to 4:0, none; 1, mild; 2, moderate; 3, significant; 4, extremely significant. The sum of the three indicators represents the severity of inflammation (score, 0-12).

Determination of TNF-α and IL-6 levels in serum: same as Example 3.

Preparation of tissue sections and H&E staining: same as Example 3.

QPCR detection of gene expression: same as Example 2.

Data statistical analysis: same as Example 2.

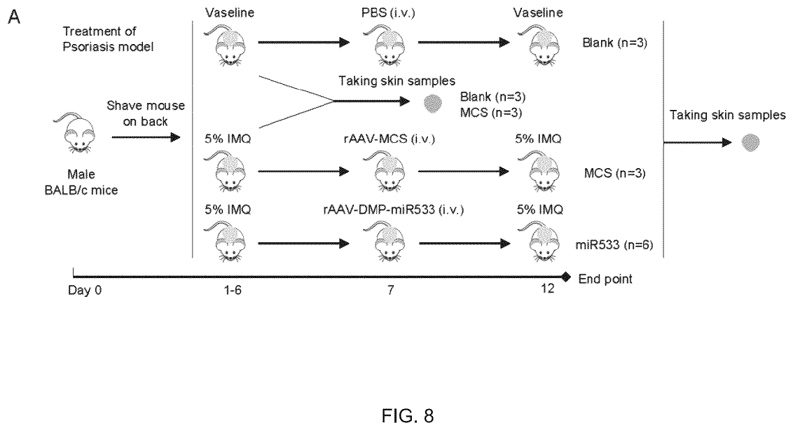

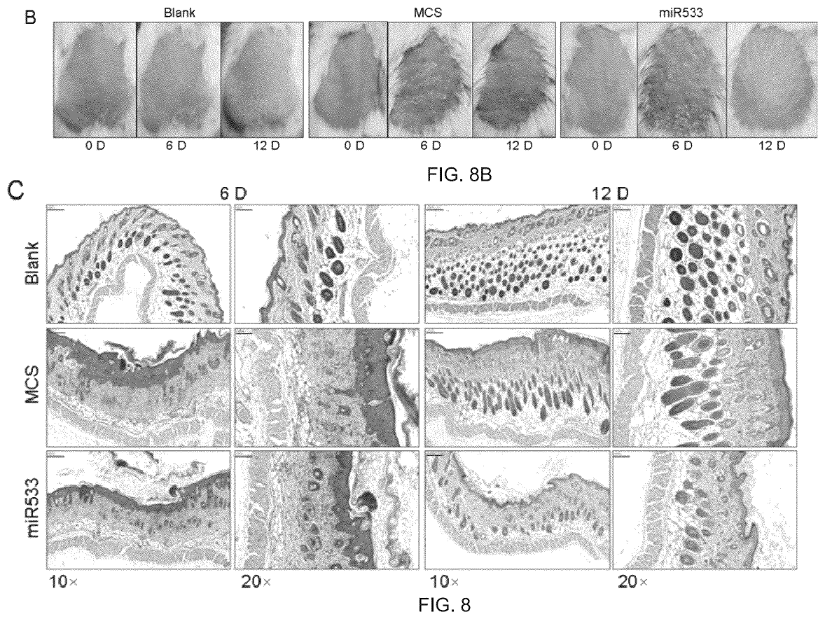

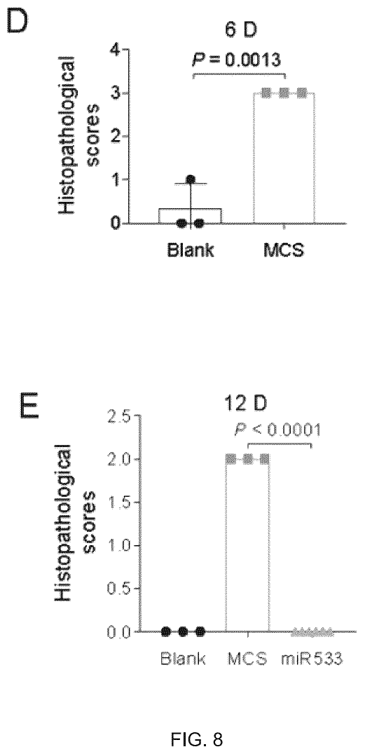

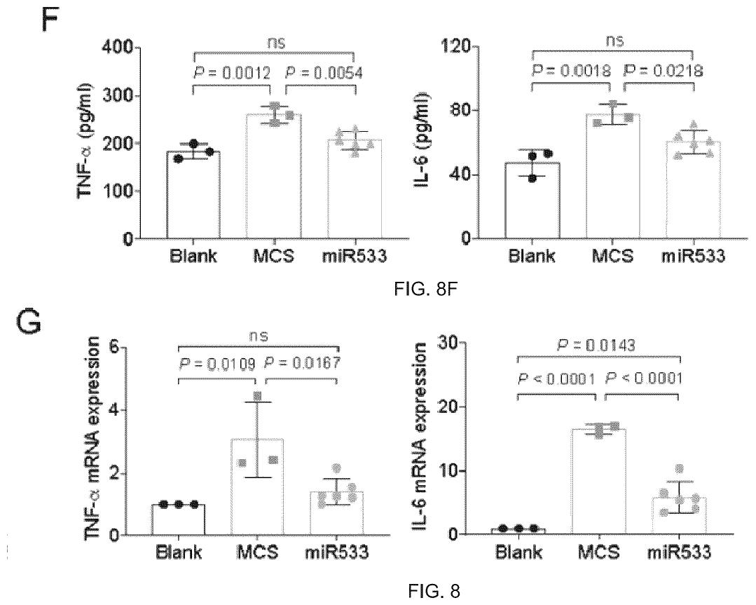

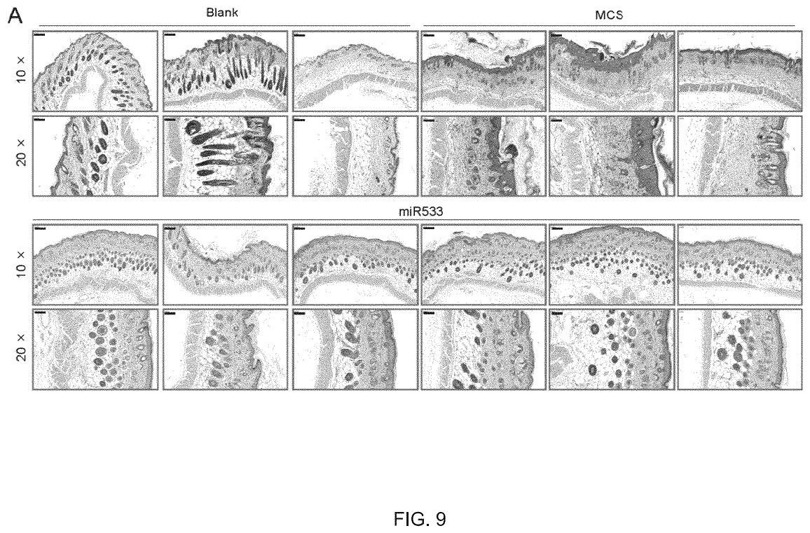

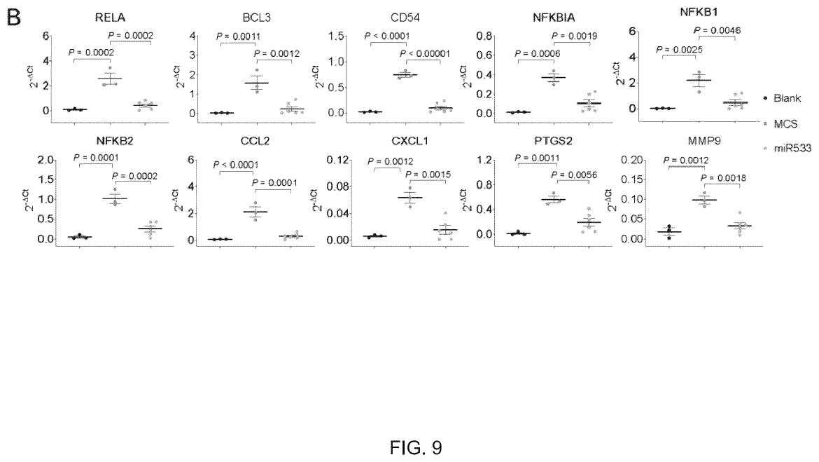

Results: to further confirm the anti-inflammatory effect of rAAV-DMP-miR533 in vivo, the mouse psoriasis model was induced by imiquimod (IMQ) and treated with various agents (FIG. 8A). IMQ was applied continuously on the shaved back skin of mice, and 6 days later, the mice showed symptoms such as redness, inflammation, itching, skin thickening, and silver scales (FIG. 8B). Then PBS, rAAV-MCS, and rAAV-DMP-miR533 were intravenously injected into the IMQ-induced psoriasis mice. The results showed that rAAV-MCS treatment had no effect on the recovery of pathological damage; while rAAV-DMP-miR533 treatment made the damaged skin of mice significantly close to that of healthy mice treated with PBS (healthy group) (FIG. 8B). H&E staining of skin tissue sections also showed that IMQ-induced mouse skin had obvious pathological features such as abscesses, hyperkeratosis, and inflammatory cell infiltration; while rAAV-DMP-miR533 treatment could significantly restore the skin (FIGS. 8C, 8D, and 8E; FIG. 9A). Detection of serum proinflammatory factors showed that TNF-α and IL-6 levels increased after IMQ induction (FIG. 8F). However, rAAV-DMP-miR533 treatment significantly reduced the levels of TNF-α and IL-6 I in serum (FIG. 8F). Detection of gene expression in the skin showed that IMQ significantly activated the expression of TNF-α, IL-6, NF-κB RELA and target genes thereof (FIGS. 8G and 9B). However, after rAAV-DMP-miR533 treatment, these genes were significantly inhibited (FIGS. 8G and 9B). These data indicate that intravenous injection of rAAV-DMP-miR533 has a good therapeutic effect on psoriasis in mice.

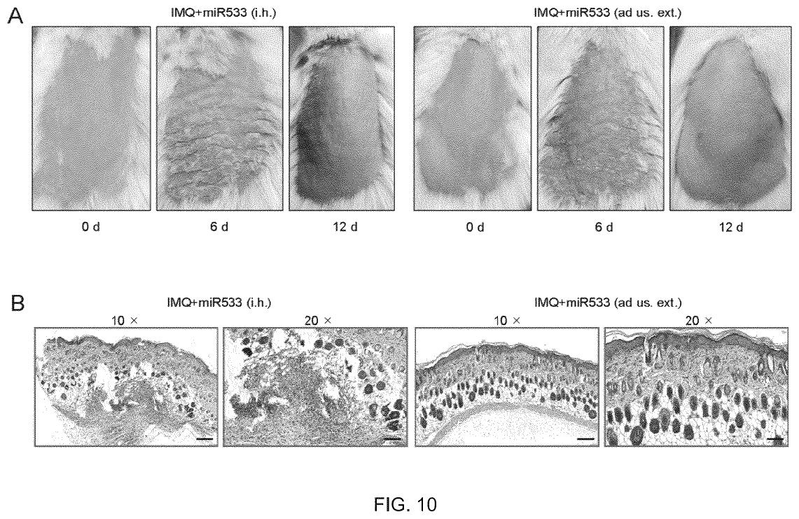

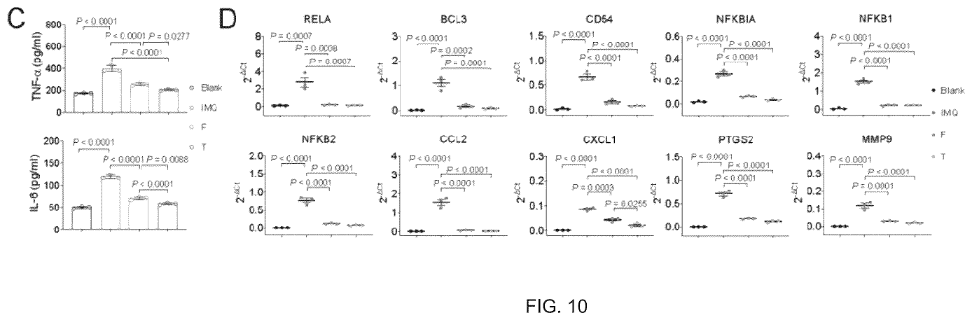

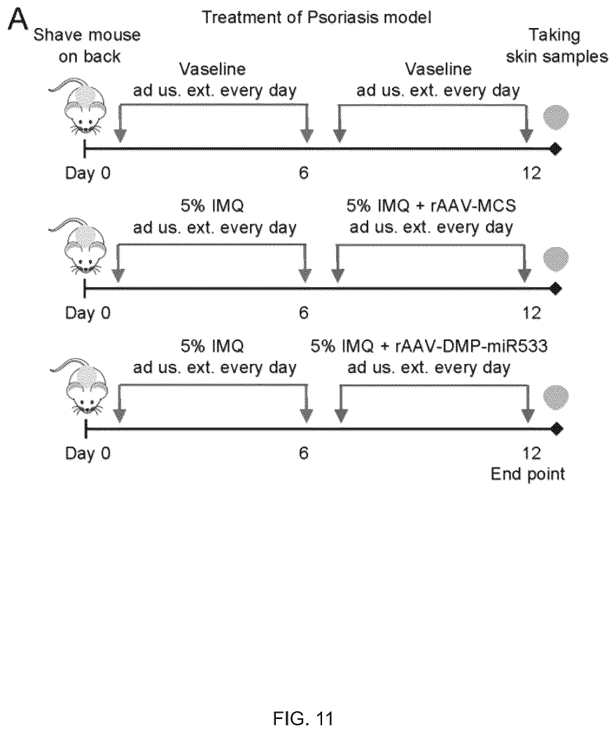

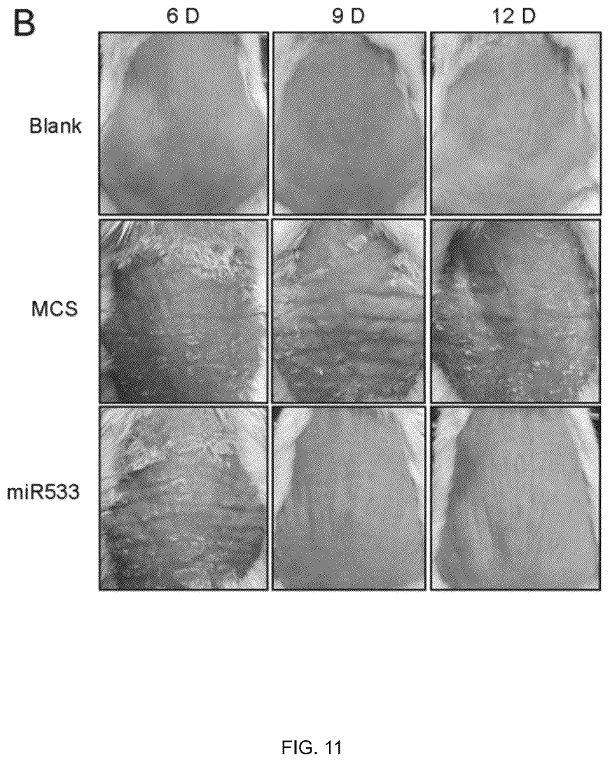

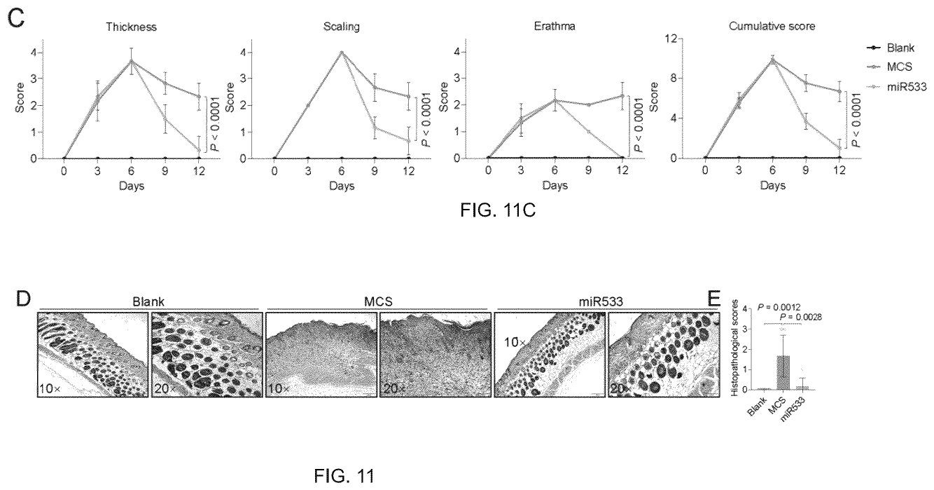

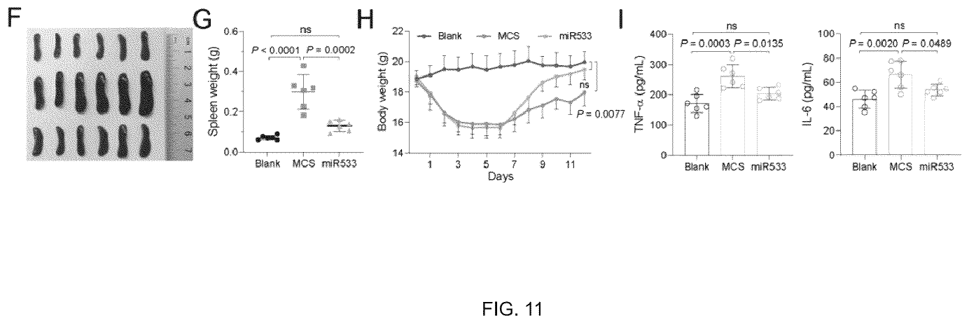

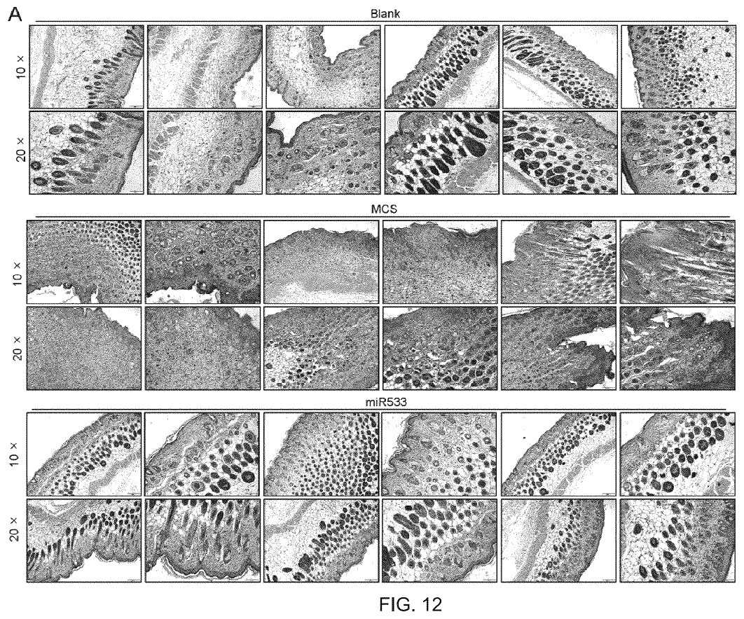

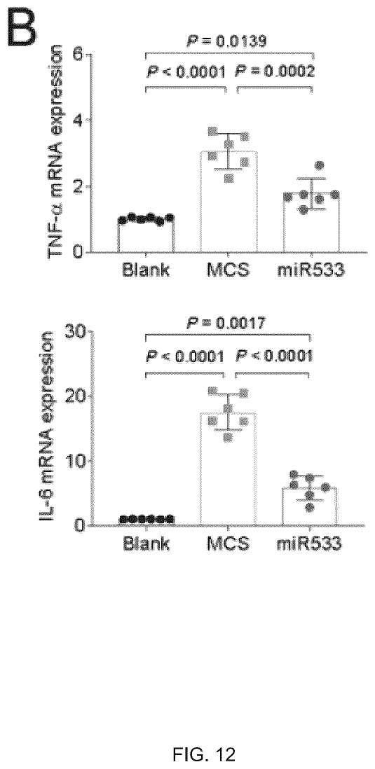

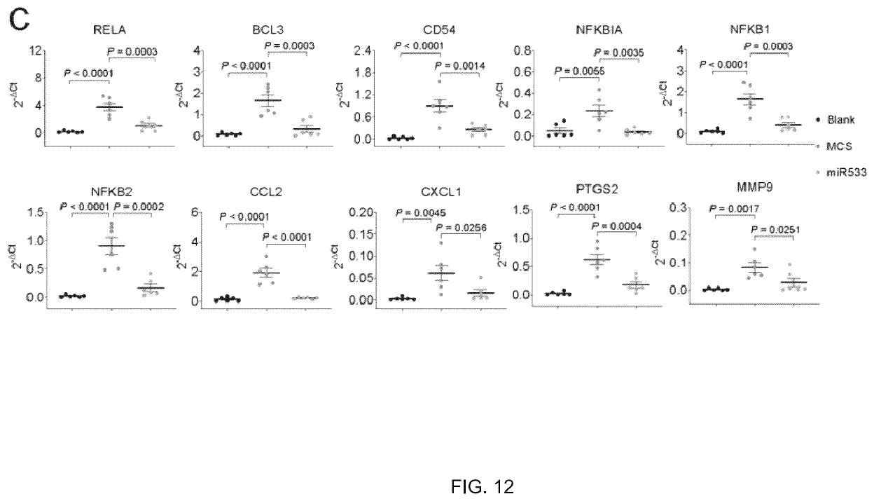

In order to find other administration methods, a mouse with IMQ-induced psoriasis was treated with subcutaneous injection and skin application. The results showed that both administration methods achieved similar therapeutic effects as the above intravenous injection (FIG. 10), including skin recovery (FIGS. 10A and 10B), serum proinflammatory factors (FIG. 10C), and downregulation of the expression of NF-κB RELA and target genes thereof (FIG. 10D). Considering the better therapeutic effect and convenience of administration, the skin application method was selected for scaled-up treatment, including more mice (n=6) (FIG. 11A). Psoriasis mice were treated with petrolatum and rAAV-DMP-miR533 mixed in petrolatum for 6 consecutive days (FIG. 11A). The results showed that the rAAV-DMP-miR533 skin application method achieved good therapeutic effects, including restoration of skin appearance (FIG. 11B), low psoriasis area and severity index (PASI) (FIG. 11C), healing of skin tissue structure (FIGS. 11D and 11E; FIG. 12A), improvement of splenomegaly (FIGS. 11F and 11G), weight gain (FIG. 11H), decreased serum TNF-α and IL-6 levels (FIG. 11I), and significant downregulation of TNF-α, IL-6 (FIG. 12B) and NF-κB RELA and target genes thereof in skin tissues (FIG. 12C). In summary, these results fully demonstrated that the rAAV-DMP-miR533 has good in vivo anti-inflammatory efficacy in IMQ-induced psoriasis mice.

### Example 5

### Construction and Treatment of Arthritis Model

30 male DBA/1J mice (8 weeks; Cavens, China) were randomly divided into 5 groups. One of the 5 groups was injected with PBS alone (n=6) as a healthy control. A collagen-induced arthritis (CIA) mouse model was established by double immunization. For the first immunization, the mice were intradermally injected with equal volumes of a chicken type II collagen solution (2 mg/mL) and a complete Freund's adjuvant (2 mg/mL) (Chondrex, Redmond, WA, USA) in the tail. 21 days after the first immunization, the chicken type II collagen solution was emulsified with an incomplete Freund's adjuvant (Chondrex, Redmond, WA, USA) and boosted in the tail of the mice at a different location from the first immunization. All paws of each mouse were scored to characterize the disease: 0, normal; 1, mild swelling and erythema limited to the midfoot and ankle; 2, mild swelling and erythema extending to the midfoot and ankle; 3, moderate swelling and erythema from the metatarsal joint to the ankle; 4, severe swelling and erythema of the foot, ankle, and fingers. The clinical score for each mouse was the sum of the scores of the four paws.

CIA mice were divided into 4 experimental groups: (1) CIA group: CIA mice were injected with PBS as a control group (iv; n=6); (2) MTX treatment group: CIA mice were injected with MTX (1 mg/kg) every other day for 6 times (iv; n=6); (3) NT treatment group: CIA mice were intravenously injected with rAAV-NT (1×10⁹ vg/mouse; n=6) every other day for 3 times; (4) miR533 treatment group: CIA mice were intravenously injected with rAAV-DMP-miR533 (1×10⁹ vg/mouse; n=6) every other day for 3 times. The body weight and clinical scores of the mice were monitored and evaluated every other day. The ankle width, paw thickness, and tail thickness of mice were measured with a vernier caliper. All mice were euthanized and photographed 4 weeks after MTX, NT, or miR533 administration. Serum samples from each group were collected for biochemical index detection. ELISA kits were used to detect the expression levels of TNF-α and IL-6 in the hind paws and serum samples. Tissues including heart, liver, spleen, lung, and kidney were collected for H&E staining analysis. The spleens of all mice were photographed and weighed. Ankle joint tissues were used for subsequent H&E staining analysis and gene expression detection. Histopathological scoring was blindly performed by other unrelated technicians according to the following four levels: 0, normal synovium; 1, synovial hypertrophy and cell invasion; 2, pannus and cartilage erosion; 3, cartilage and subchondral bone erosion; 4, dysfunction and stiffness of the entire joint.

Determination of TNF-α and IL-6 levels in serum: same as Example 3.

Preparation of tissue sections and H&E staining: same as Example 3. In this experiment, mouse paw (including ankle joints) tissues were additionally dissected.

Preparation of tissue sections and H&E staining: same as Example 2.

QPCR detection of gene expression: same as Example 2.

Data statistical analysis: same as Example 2.

Micro-CT: all DBA/1J mice were killed and their paws (including ankle joints) were collected for micro-CT imaging (Micro-CT) using an in vivo micro-CT scanner (vivaCT 80, SCANCO Medical AG, Switzerland). Reconstruction and analysis of high-resolution tomographic images were performed on the SCANCO GPU Accelerated Reconstruction system.

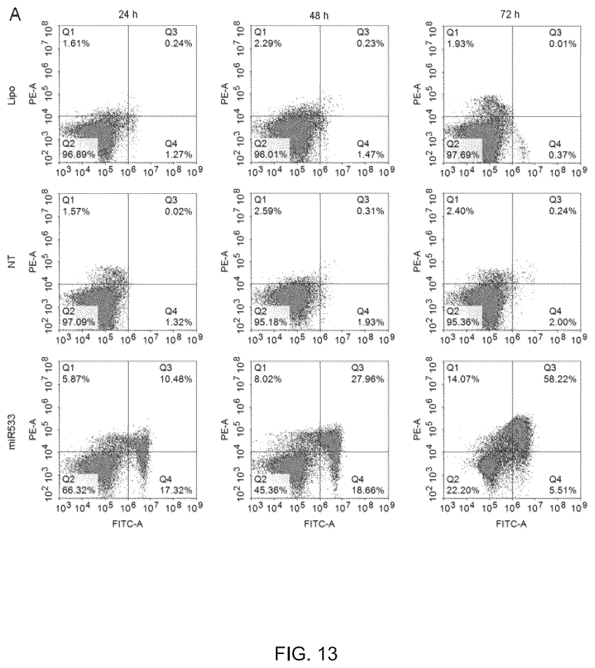

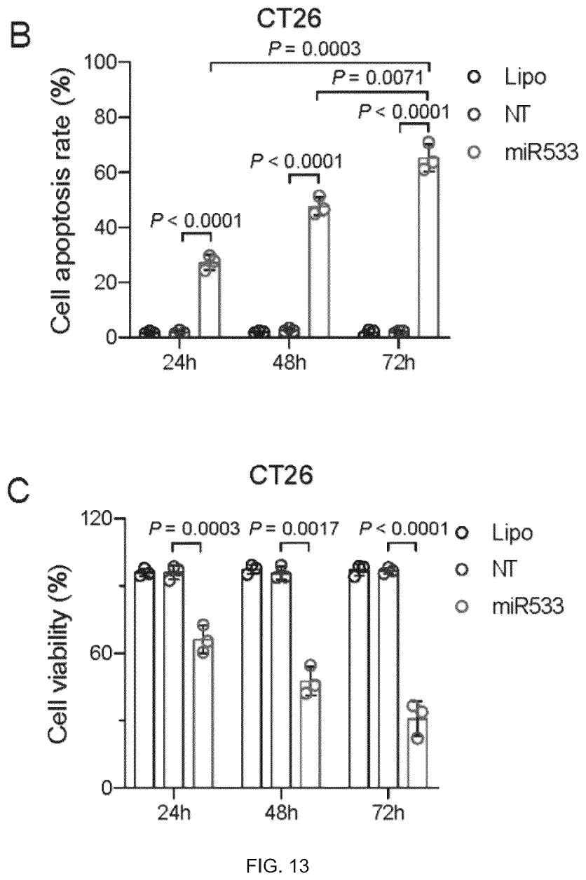

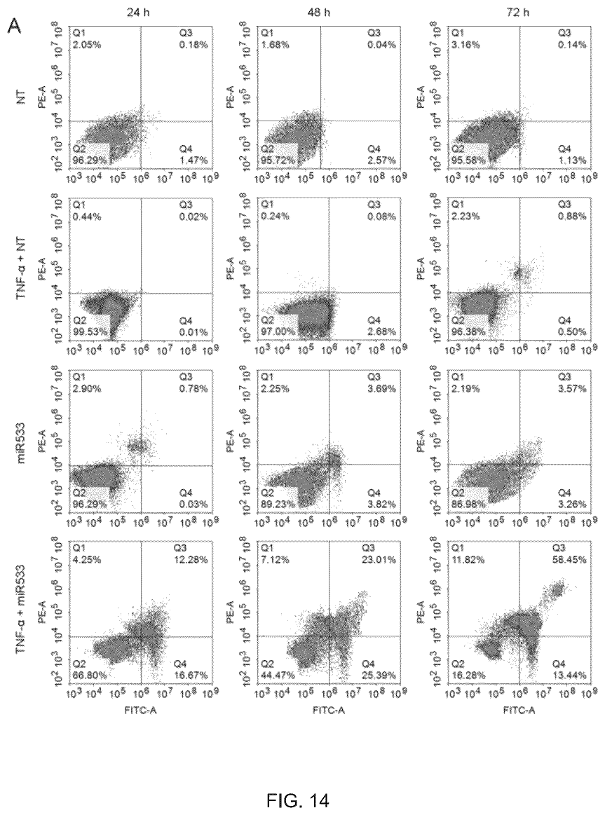

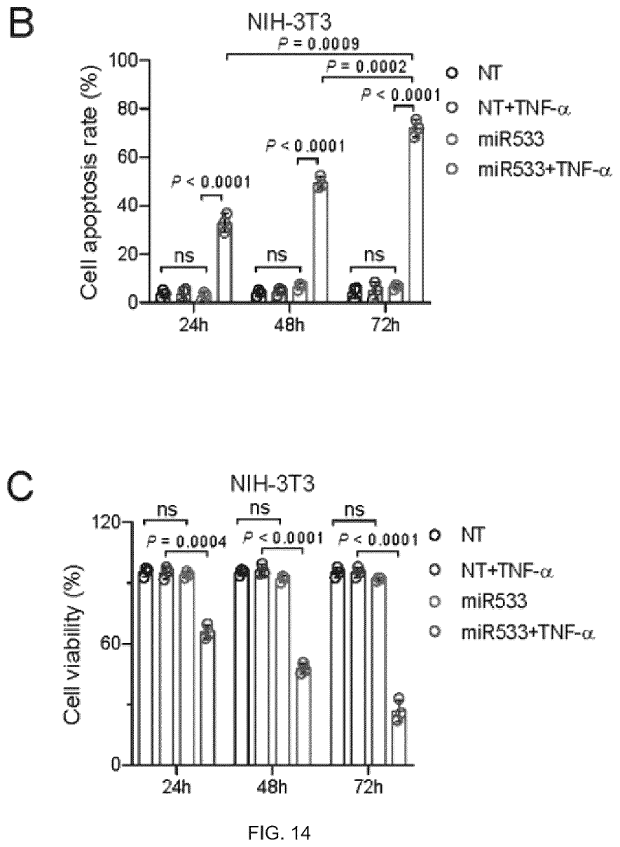

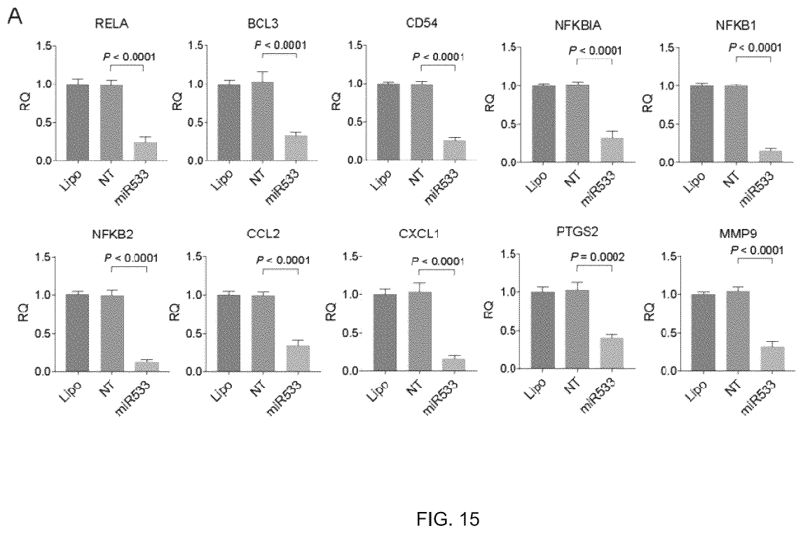

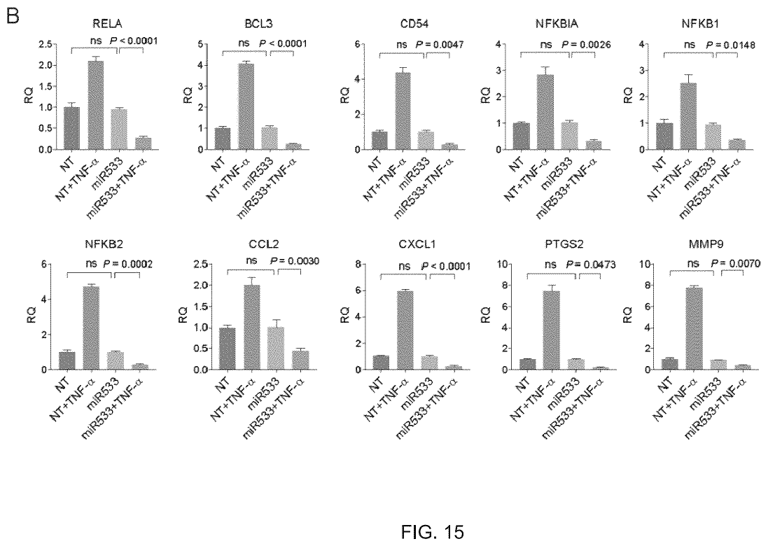

Results: in the above cell and mouse experiments, pAAV-MCS and rAAV-MCS were used as negative controls for pAAV-DMP-miR533 and rAAV-DMP-miR533, respectively. In order to provide a more suitable negative control for DMP-miR533, a new vector DMP-NT was constructed, which encodes a microRNA with no target (NT) for both the human and mouse genomes. To evaluate the vector, mouse colon cancer cells (CT26) were transfected with the pAAV-DMP-miR533 and the pAAV-DMP-NT, respectively. The results showed that the pAAV-DMP-miR533 significantly inhibited the cell growth of CT26 and induced cell apoptosis (FIGS. 13A-13C); while the pAAV-DMP-NT had no significant effect on cell growth and apoptosis (FIGS. 13A-13C). To further evaluate the two vectors, normal mouse embryonic fibroblasts (NIH-3T3) were transfected with the two vectors. The results showed that neither vector induced significant apoptosis and growth inhibition in cells (FIGS. 14A-14C). However, when cells were induced by TNF-α, the pAAV-DMP-miR533 induced significant apoptosis and growth inhibition in cells (FIGS. 14A-14C). The pAAV-DMP-NT still had no effect on cells induced by TNF-α (FIGS. 14A-14C). QPCR detection showed that the pAAV-DMP-miR533 significantly inhibited the expression of NF-κB RELA and target genes thereof in NIH-3T3 induced by CT26 and TNF-α (FIGS. 15A and 15B); while the pAAV-DMP-NT had no effect on gene expression in both cells (FIGS. 15A and 15B). In summary, these results indicate that the DMP-miR533 produces an in vitro anti-inflammatory effect by inhibiting NF-κB activity. In addition, non-cytotoxic pAAV-DMP-NT was packaged into AAV to prepare the rAAV-DMP-NT, and the rAAV-DMP-NT was used as the corresponding negative control to further evaluate the in vivo anti-inflammatory effect of the rAAV-DMP-miR533.

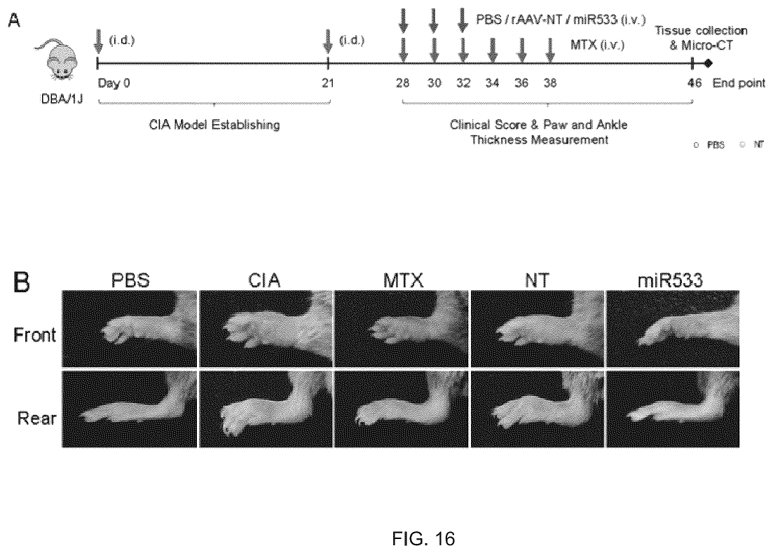

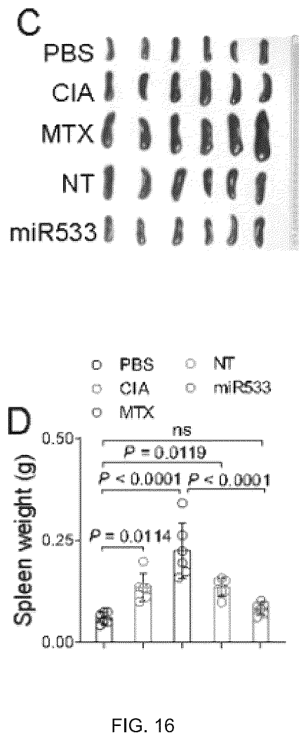

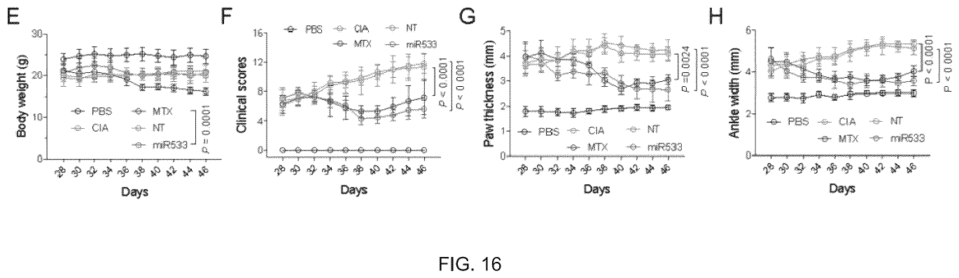

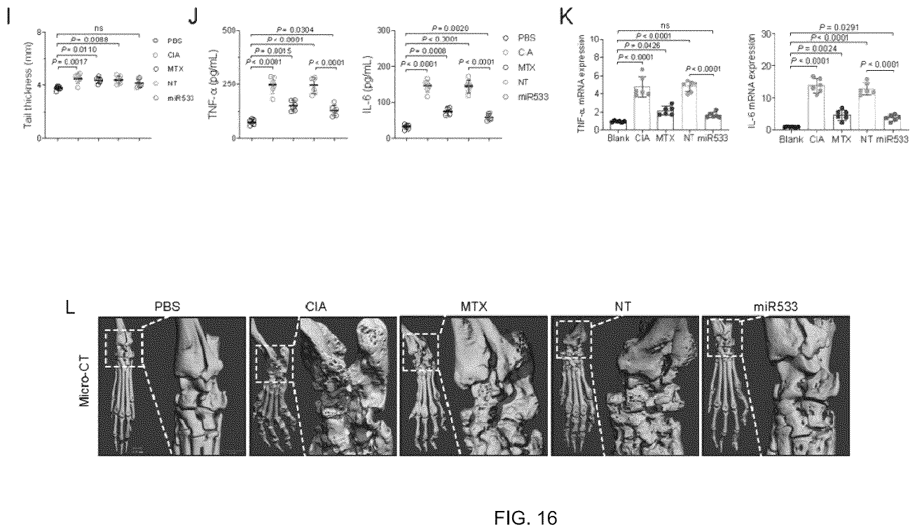

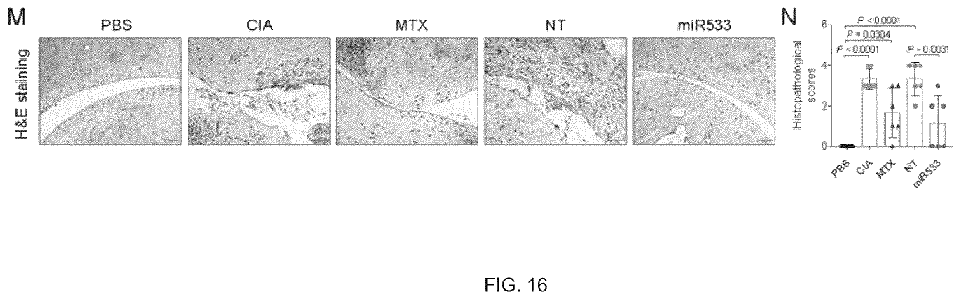

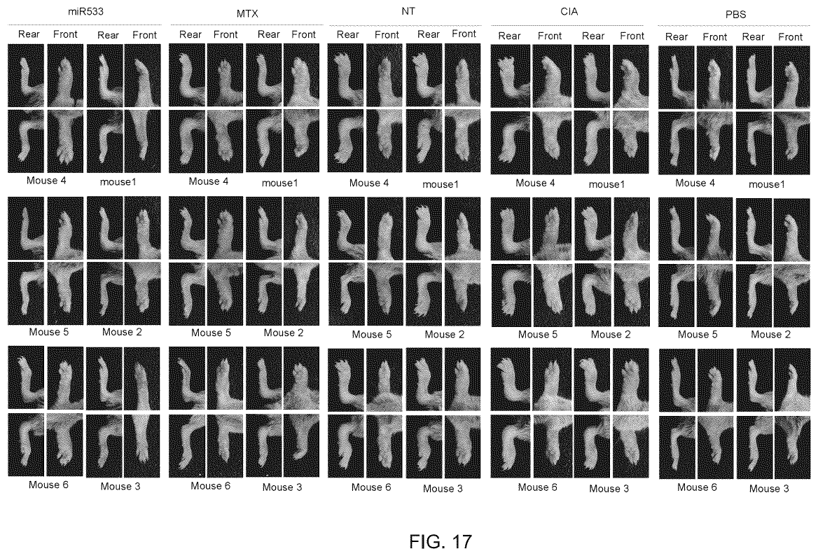

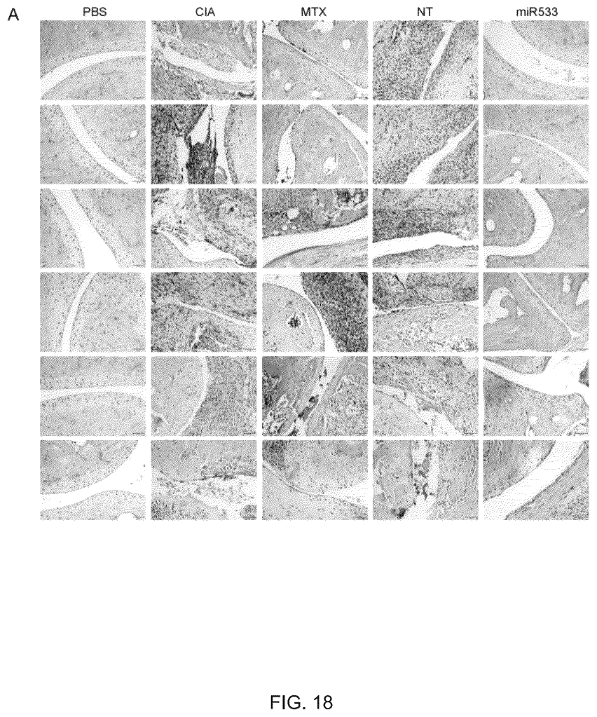

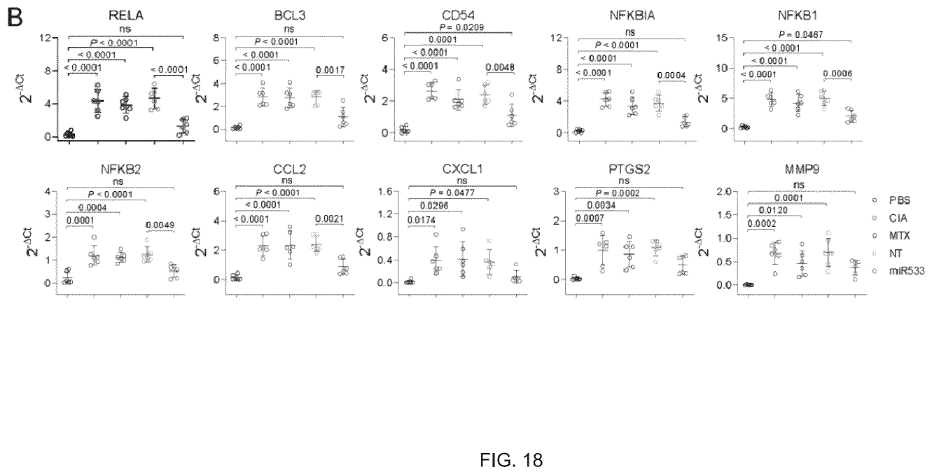

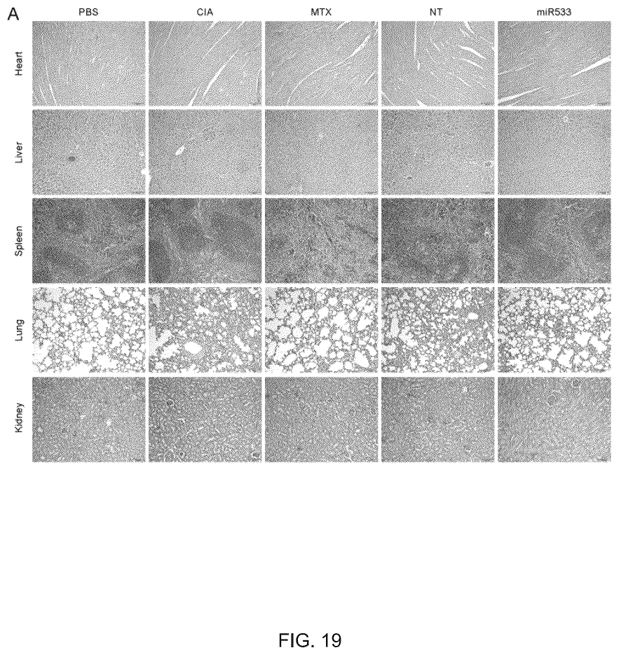

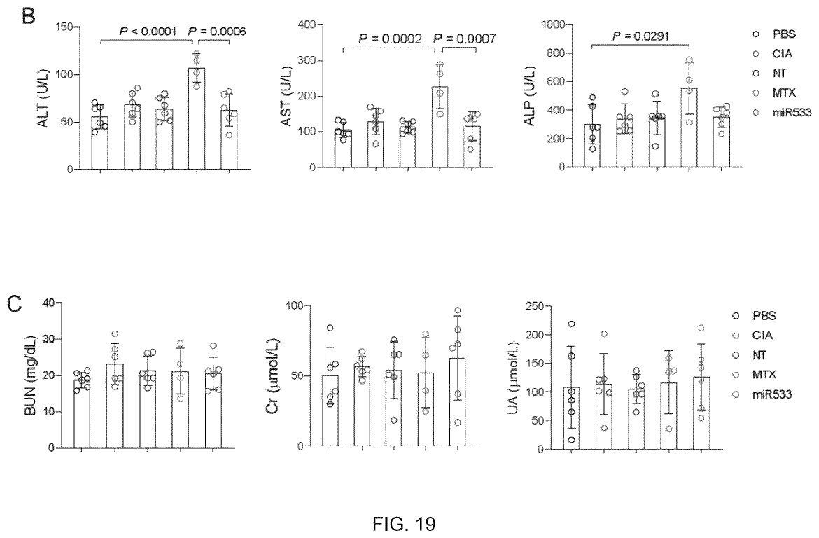

Rheumatoid arthritis (RA) is a chronic autoimmune disease characterized by joint lesions. The collagen-induced arthritis (CIA) mouse model was established by double immunization for RA treatment studies (FIG. 16A). On 28th day after the first immunization, the CIA mice were randomly divided into 4 groups (n=6) and intravenously injected with PBS, a methotrexate solution (MTX), the rAAV-DMP-NT, and the rAAV-DMP-miR533. The healthy group mice were intravenously injected with PBS only as a healthy control (n=6). On 46th day, all mice were euthanized. Imaging of the paws (including the ankle joints) showed that the rAAV-DMP-miR533 achieved a better therapeutic effect than MTX (FIGS. 16B and 17). Meanwhile, rAAV-DMP-miR533 treatment restored the normal spleen volume and weight of the CIA mice, while the spleen enlargement of the mice treated with MTX was more severe than that of the CIA model mice (FIGS. 16C and 16D). In addition, the body weight of the mice remained stable after rAAV-DMP-miR533 treatment, while MTX treatment caused the mice to lose weight (FIG. 16E). Dynamic measurement of pathological changes in the mice showed that both rAAV-DMP-miR533 and MTX treatment significantly improved clinical scores (FIG. 16F), paw thickness (FIG. 16G), and ankle width (FIG. 16H). It should be noted that only rAAV-DMP-miR533 treatment made the tail width of the mice close to that of the healthy mice (FIG. 16I). In the CIA model group, serum levels of proinflammatory factors TNF-α and IL-6 were significantly increased; both rAAV-DMP-miR533 and MTX significantly reduced the levels of TNF-α and IL-6 in serum (FIG. 16J). However, the rAAV-DMP-miR533 had a better therapeutic effect than MTX (FIG. 16J). These therapeutic effects were also confirmed by the expression levels of mRNA of TNF-α and IL-6 in the hind paw tissues (FIG. 16K). More convincingly, Micro-CT imaging of the mouse hind paws showed that the ankle and finger joints of the CIA mice had severe bone erosion (FIG. 16L). After rAAV-DMP-miR533 treatment, the bone erosion of the mice was greatly improved, and the therapeutic effect was significantly better than MTX (FIG. 16L). The results of H&E staining and joint histopathological evaluation also supported this conclusion (FIGS. 16M and 16N; FIG. 18A). The CIA mice have pathological features such as a large number of pannus, severe bone destruction, extensive cartilage damage, and inflammatory cell infiltration. Both rAAV-DMP-miR533 and MTX treatment significantly improved these pathological changes, and rAAV-DMP-miR533 treatment was more prominent. In addition, rAAV-DMP-miR533 treatment also significantly inhibited the expression of NF-κB RELA and target genes thereof in the forepaw (FIG. 18B). MTX treatment did not regulate the expression of these genes (FIG. 18B), indicating that inflammatory cells still existed. It is worth noting that in all the above experiments, the rAAV-DMP-NT did not show a therapeutic effect (FIGS. 16 and 18). H&E section staining of major organs (heart, liver, spleen, lungs, and kidneys) showed that the rAAV-DMP-miR533 significantly improved tissue damage caused by CIA modeling, especially lung damage (FIG. 19A), while MTX was not significant, and even caused some damage and necrosis to the liver and spleen (FIG. 19A). Finally, the serum collected on the 46th day was tested for biochemical indices, and the data further showed that the rAAV-DMP-miR533 had no effect on these biochemical indices and had good biosafety (FIG. 19B). After MTX treatment, ALT, AST and ALP in mouse serum generally increased, indicating that the MTX was hepatotoxic (FIG. 19C). In summary, these results demonstrate that the rAAV-DMP-miR533 has a good in vivo anti-inflammatory effect in the collagen-induced arthritis mice.

### Example 6

### Anti-Inflammatory Effect of Multiple Copies of DMP-miR533

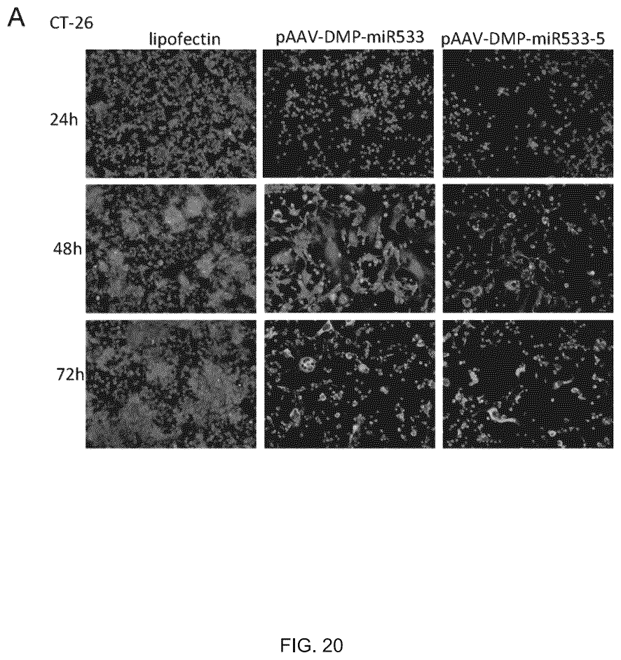

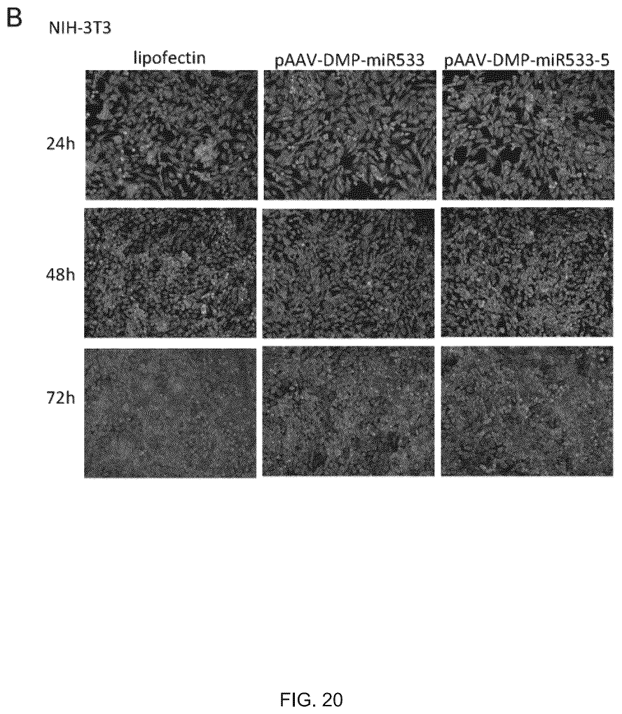

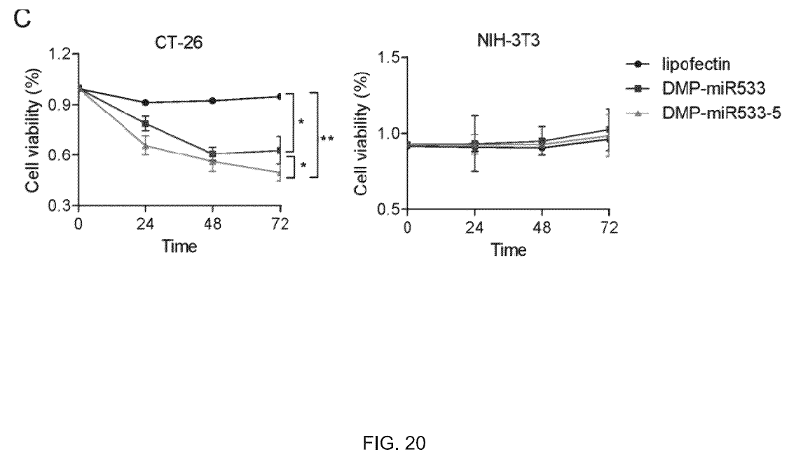

In order to further explore the effect of increasing the copy count of DMP-miR533 on the anti-inflammatory effect of rAAV-DMP-miR533, a rAAV packaging plasmid pAAV-DMP-miR533-5 containing 5 copies of DMP-miR533 was constructed. CT-26 and NIH-3T3 were transfected in parallel with the plasmid pAAV-DMP-miR533-5 and the rAAV packaging plasmid pAAV-DMP-miR533 containing a single copy of DMP-miR533. The transfection method was the same as in Example 2. The effects of the two plasmid transfections on the apoptosis and viability of the two cells were observed. The results showed that compared with the pAAV-DMP-miR533, the pAAV-DMP-miR533-5 further significantly improved the pro-apoptotic effect on inflammatory cells CT-26 and inhibited the growth activity of inflammatory cells (FIG. 20), while still having no significant effect on the apoptosis and viability of normal cells NIH-3T3 (FIG. 20). It can be inferred that the rAAV-DMP-miR533 virus containing 5 copies of DMP-miR533 packaged with pAAV-DMP-miR533-5 should have a better anti-inflammatory effect in vivo.

1-9. (canceled)

10. A recombinant adeno-associated virus for treating an inflammatory disease, comprising one or more copies of a functional DNA fragment DMP-miR533, wherein
the functional DNA fragment DMP-miR533 is composed of two functional elements DMP and miR533, wherein the DMP is an NF-κB specific promoter, and the miR533 is a microRNA that targets an NF-κB mRNA.

11. The recombinant adeno-associated virus of claim 10, wherein the DMP includes an NF-κB decoy and a minimal promoter.

12. The recombinant adeno-associated virus of claim 10, wherein the DMP has a sequence shown in SEQ ID NO: 1.

13. The recombinant adeno-associated virus of claim 10, wherein the miR533 encodes an artificial microRNA targeting an NF-κB family member RELA, and the miR533 has a sequence shown in SEQ ID NO: 2.

14. The recombinant adeno-associated virus of claim 10, wherein after the functional DNA fragment DMP-miR533 is introduced into a cell by the recombinant adeno-associated virus, the functional element DMP is capable of binding to a transcription factor protein NF-κB in a nucleus of the cell to activate the expression of miR.

15. The recombinant adeno-associated virus of claim 10, wherein the expressed miR533 is capable of binding to the NF-κB mRNA in a cytoplasm after being processed and matured by an intracellular microRNA maturation system, thereby inhibiting the expression of an NF-κB protein.

16. The recombinant adeno-associated virus of claim 10, wherein the adeno-associated virus includes any one of various serotypes of adeno-associated viruses AAV1-AAV9.

17. The recombinant adeno-associated virus of claim 10, wherein the recombinant adeno-associated virus comprises 5 copies of the functional DNA fragment DMP-miR533.

18. The recombinant adeno-associated virus of claim 10, wherein the inflammatory disease includes inflammation caused by infection, spontaneous inflammation, an autoimmune disease, a neurodegenerative disease, skin inflammation, or cancer.

19. The recombinant adeno-associated virus of claim 18, wherein the inflammatory disease includes acute colitis, psoriasis, or arthritis.

20. A medical composition for treating an inflammatory disease, comprising the recombinant adeno-associated virus of claim 10.

21. The medical composition of claim 20, wherein the inflammatory disease includes inflammation caused by infection, spontaneous inflammation, an autoimmune disease, a neurodegenerative disease, skin inflammation, or cancer.

22. The medical composition of claim 20, further comprising petrolatum for treating skin inflammation.

23. The medical composition of claim 22, wherein the skin inflammation includes psoriasis.

24. A method of treating a subject suffering from an inflammatory disease, comprising administering to the subject an effective amount of the recombinant adeno-associated virus of claim 10.

25. A method of treating a subject suffering from a skin inflammation, comprising administering to the subject an effective amount of the medical composition of claim 22.

26. The method of claim 25, wherein the medical composition is administered onto a skin of the subject.

27. The method of claim 25, wherein the inflammatory disease includes psoriasis.

28. The method of claim 25, wherein the inflammatory disease includes arthritis.

29. A method of constructing the recombinant adeno-associated virus of claim 10, comprising:
(1) amplifying the DMP-miR533 from a vector pDMP-miR533 and ligating the DMP-miR533 to a vector pAAV-MCS to construct pAAV-DMP-miR533; and
(2) transfecting a 293T cell with the pAAV-DMP-miR533 and two auxiliary plasmids, pAAV-Helper and pAAV-RC;
after cell culture, collecting the cell and a culture medium culturing the cell and freeze-thawing the cell and the culture medium, adding pure chloroform to a lysate of the freeze-thawed cell to obtain a mixture and shaking the mixture;
adding NaCl to the mixture, shaking the mixture until the NaCl is dissolved, and collecting a supernatant after centrifugation;
adding PEG8000 to the supernatant, shaking it until the PEG8000 is dissolved, and discarding a supernatant after centrifugation, dissolving a precipitate, adding DNase and RNase to the dissolved precipitate to react and incubating at a room temperature to obtain a reactant, extracting the reactant to collect an aqueous phase containing a purified virus, and
after the virus is quantified, segmenting the aqueous phase containing the purified virus and storing at −80° C. for later use, wherein the obtained virus is named as the rAAV-DMP-miR533.
